(19) United States
(12) Patent Application Publication
(10) Pub. No.: US 2019/0024075 A1
(43) Pub. Date: Jan. 24, 2019

(54) METHODS AND COMPOSITIONS FOR TARGET DETECTION

(71) Applicant: Arc Bio, LLC, Cambridge, MA (US)

(72) Inventors: Stephane B. GOURGUECHON, San Mateo, CA (US); Meredith L. CARPENTER, San Mateo, CA (US); Eric HARNESS, Sunnyvale, CA (US)

(21) Appl. No.: 16/079,014

(22) PCT Filed: Feb. 23, 2017
(86) PCT No.: PCT/US17/19212

§ 371 Date: Aug. 22, 2018

Related U.S. Application Data
(60) Provisional application No. 62/298,937, filed on Feb. 23, 2016.

(51) Int. Cl. C12N 15/10; C12N 15/63; C12N 9/16; C12Q 1/6816; C12Q 1/686

(52) U.S. Cl. CPC C12N 15/102; C12N 15/63; C12N 9/16; C12Q 1/6816; C12N 2310/20; C12Q 2521/301; C12Q 2563/173; C12Q 2565/531; C12Q 1/686

(57) ABSTRACT

Provided herein are methods and compositions for the detection and identification of targets in a sample, using target-specific guide nucleic acids and nucleic acid-guided nuclease system proteins.

### CROSS-REFERENCE

This application claims the priority benefit of U.S. Provisional Patent Application Ser. No. 62/298,937, filed on Feb. 23, 2016, which is hereby incorporated by reference in its entirety.

### BACKGROUND

Nucleic acid-based detection methods should be cost-effective, fast, sensitive, and accurate. The detection platform should also be simple to use and interpret, stable under a wide range of operating conditions (such as temperature, humidity, lighting conditions, and access to infrastructure), preferably portable and disposable. (Yager et al., Nature, 2006, 442, 412-418). Furthermore, they should provide the required sensitivity and specificity. (Weigl et al, Lab Chip, 2008, 8, 1999-2014). The ability to perform multiplex tests is another important prerequisite for detection methods and devices.

One application for the use of nucleic acid sequence detection, is for detection of pathogens. Infectious diseases remain a major cause of morbidity and mortality throughout the world. Conventional and standard methods of pathogen detection include cell culture, PCR, and enzyme immunoassay, which are often labor-intensive and can take from several hours to days to perform. (Foudeh et al., Lab Chip, 2012, 12, 3249-3266). In the World Health Organization's 2004 report, infectious diseases were identified as the second leading cause of mortality throughout the world after cardiovascular disease (WHO, The World Health Report 2004, Genève, 2004). This problem is particularly magnified in area of poor hygiene, with limited access to centralized labs for diagnostics and treatments. Even in industrialized nations, there remain issues to be addressed with respect to food industries, pathogen outbreaks, and sexually transmitted diseases. In the political sphere, the threats of biological warfare are also a real possibility. Effective pathogen detection and identification is critical for the prevention and treatment of infectious diseases.

There are other applications for a nucleic acid sequence detection method, in addition to pathogen detection and identification. For example, detection of a plant from which a toxin is derived (e.g., castor oil plant containing ricin), or single nucleotide polymorphism (SNP) detection for the purposes of human identification or tumor sequencing, are but two examples.

Therefore, there is a need in the art to provide a cost-effective, fast, sensitive, and accurate method of nucleic acid detection and identification that can be carried in out in a range of operating conditions. Provided herein are methods and compositions that address this need.

### SUMMARY

Provided herein are methods and compositions for the detection and identification of target nucleic acids, using target-specific guide nucleic acid (gNA) mediated nuclease system proteins, such as guide RNA (gRNA) mediated CRISPR/Cas system proteins.

In one aspect, provided herein is a method of identifying a target in a sample comprising: (a) contacting nucleic acid from a sample with a plurality of gNA-nucleic acid-guided nuclease system protein complexes, wherein the complexes are targeted to at least one target, and wherein the nucleic acid, the gNA-nucleic acid-guided nuclease system protein complexes, or both the nucleic acid and the gNA-nucleic acid-guided nuclease system protein complexes comprise a label; and (b) identifying the target in the sample, wherein the identifying is achieved by detecting a specific signal from the label, wherein the presence of a specific signal indicates binding of the gNA-nucleic acid-guided nuclease system protein complex to the nucleic acid. In some embodiments, nucleic acid comprises a label. In some embodiments, the nucleic acid label is an intercalating label. In some embodiments, the nucleic acid is labeled prior to the contacting step. In some embodiments, the nucleic acid is labeled after the contacting step. In some embodiments, a plurality of the gNA-nucleic acid-guided nuclease system protein complexes comprise a label. In some embodiments, a plurality of the gNA-nucleic acid-guided nuclease system protein complexes are labeled prior to the contacting step. In some embodiments, a plurality of the gNA-nucleic acid-guided nuclease system protein complexes are labeled after the contacting step. In some embodiments, the nucleic acid and the plurality of the gNA-nucleic acid-guided nuclease system protein complexes are labeled; in some embodiments, they are both labeled prior to the contacting step; and in some embodiments, they are both labeled after the contacting step. In some embodiments, the nucleic acid is labeled prior to contacting, and the plurality of the gNA-nucleic acid-guided nuclease system protein complexes are labeled after the contacting step. In some embodiments, the nucleic acid is labeled after contacting step, and the plurality of the gNA-nucleic acid-guided nuclease system protein complexes are labeled before the contacting step. In some embodiments, the nucleic acid comprises a first label and the gNA-nucleic acid-guided nuclease system protein complexes comprise a second label, wherein the first and second label comprise a donor/acceptor pair for fluorescent resonance energy transfer (FRET). In some embodiments, the contacting is carried out at room temperature. In some embodiments, the identifying comprises detecting a single signal. In some embodiments, the identifying comprises detecting multiple signals. In some embodiments, the nucleic acid-guided nuclease system protein is a CRISPR/Cas system protein. In some embodiments, the CRISPR/Cas system protein is selected from the group consisting of Cas9, CasX, CasY, Cpf1, Cas3, Cas8a-c, Cas10, Cse1, Csy1, Csn2, Cas4, Csm2, and Cm5. In some embodiments, the nucleic acid-guided nuclease system protein is a dead nucleic acid-guided nuclease system protein. In some embodiments, the CRISPR/Cas system protein is a dead CRISPR/Cas system protein. In some embodiments, the CRISPR/Cas system protein is dCas9. In some embodiments, the nucleic acid-guided nuclease system protein is a nucleic acid-guided nuclease system nickase protein. In some embodiments, the CRISPR/Cas system protein is a CRISPR/Cas system nickase protein. In some embodiments, the nucleic acid-guided nuclease system protein exhibits reduced off-target binding. In some embodiments, the label is selected from the group consisting of an enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group, an aptamer, one member of a binding pair, and combinations thereof. In some embodiments, the label is a detectable label. In some embodiments, the sample is selected from the group consisting of a clinical sample, a forensic sample, an environmental sample, a metagenomic sample, and a food sample. In some embodiments, the sample is from a human. In some embodiments, the sample is not processed prior to the contacting. In some embodiments, the nucleic acid, the gNA-nucleic acid-guided nuclease system protein complexes, or both the nucleic acid and the gNA-nucleic acid-guided nuclease system protein complexes comprise multiple labels. In some embodiments, the complexes are targeted to a plurality of targets. In some embodiments, the target is a pathogen. In some embodiments, the pathogen is selected from the pathogens in Table 1. In some embodiments, the complexes targeted to different targets comprise different labels. In some embodiments, the complexes targeted to different targets comprise the same label. In some embodiments, the gNA-nucleic acid-guided nuclease system protein complexes are attached to a substrate. In some embodiments, the nucleic acid-guided nuclease system proteins are attached to a substrate. In some embodiments, the gNAs are attached to a substrate. In some embodiments, the nucleic acid is attached to a substrate. In some embodiments, the substrate is silica, plastic, glass, or metal. In some embodiments, the substrate is a 2-dimensional substrate. In some embodiments, the substrate comprises a 3-dimensional substrate. In some embodiments, the substrate comprises a chamber or is a cylindrical array. In some embodiments, the gNAs or gNA-nucleic acid-guided nuclease system protein complexes are attached to the substrate in a known order. In some embodiments, the substrate is reusable. In some embodiments, the contacting takes place in solution. In some embodiments, the gNA-nucleic acid-guided nuclease system protein complexes, gNAs, or nucleic acid-guided nuclease system proteins are in solution. In some embodiments, the gNA-nucleic acid-guided nuclease system protein complexes comprise at least 1 unique gNA. In some embodiments, the nucleic acid is sheared. In some embodiments, the nucleic acid is amplified. In some embodiments, the nucleic acid is blocked at one or more termini with non-extendible nucleotides. In some embodiments, the nucleic acid is further analyzed by sequencing after the identifying step. In some embodiments, a plurality of targets are simultaneously identified. In some embodiments, the method is carried out in less than 24 hours. In some embodiments, the sample is subject to a prior screening step. In some embodiments, the sample is subject to multi-detection steps. In some embodiments, the binding of a gNA-nucleic acid-guided nuclease system protein complex to the nucleic acid indicates the presence of a target. In some embodiments, the lack of binding of a gNA-nucleic acid-guided nuclease system protein complex to a nucleic acid indicates the absence of a target. In some embodiments, the method further comprises generating droplets, wherein a subset of the droplets comprise a gNA-nucleic acid-guided nuclease system protein complex. In some embodiments, a subset of the droplets comprise a gNA-nucleic acid-guided nuclease system protein complex bound to the nucleic acid. In any of the embodiments provided herein, the nucleic acid from the sample comprises DNA. In any of the embodiments provided herein, the nucleic acid from the sample is DNA. In any of the embodiments provided herein, the nucleic acid from the sample comprises RNA. In any of the embodiments provided herein, the nucleic acid from the sample is RNA.

In another aspect, provided herein is a collection of guide nucleic acids (gNAs) targeted to at least one target, wherein a plurality of the gNAs comprise a label. In some embodiments, the collection is targeted to a plurality of targets. In some embodiments, the target is a pathogen. In some embodiments, the pathogen is selected from the pathogens in Table 1. In some embodiments, the gNAs targeted to different targets comprise different labels. In some embodiments, the gNAs targeted to different targets comprise the same labels. In some embodiments, the label is a detectable label. In some embodiments, the label is selected from the group consisting of an enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group, an aptamer, one member of a binding pair, and combinations thereof. In some embodiments, the gNAs comprise more than one label. In some embodiments, the gNAs are attached to a substrate. In some embodiments, the substrate is silica, plastic, glass, or metal. In some embodiments, the substrate is a 2-dimensional substrate. In some embodiments, the substrate comprises a 3-dimensional substrate. In some embodiments, the substrate comprises a chamber or is a cylindrical array. In some embodiments, the gNAs are attached to a substrate in a known order. In some embodiments, the substrate is reusable. In some embodiments, the gNAs are in solution. In some embodiments, the collection comprises at least 1 unique gNA. In some embodiments, the gNAs are complexed to nucleic acid-guided nuclease system proteins. In some embodiments, the nucleic acid-guided nuclease system protein is a CRISPR/Cas system protein. In some embodiments, the gNAs are gRNAs. In some embodiments, the CRISPR/Cas system protein is selected from the group consisting of Cas9, CasX, CasY, Cpf1, Cas3, Cas8a-c, Cas10, Cse1, Csy1, Csn2, Cas4, Csm2, and Cm5. In some embodiments, the CRISPR/Cas system protein is a dead CRISPR/Cas system protein. In some embodiments, the CRISPR/Cas system protein is a dead CRISPR/Cas system protein. In some embodiments, the CRISPR/Cas system protein is dCas9. In some embodiments, the CRISPR/Cas system protein is CRISPR/Cas 9 system nickase protein. In some embodiments, the CRISPR/Cas system protein is CRISPR/Cas 9 system nickase protein. In some embodiments, the nucleic acid-guided nuclease system protein exhibits reduced off-target binding. In some embodiments, the nucleic acid-guided nuclease system proteins also comprise a label.

In another aspect, provided herein is a collection of gNA-nucleic acid-guided nuclease system protein complexes, wherein the gNAs are targeted to at least one target, and wherein a plurality of the complexes comprise a label. In some embodiments, a plurality of the gNAs comprise a label. In some embodiments, a plurality of the nucleic acid-guided nuclease system proteins comprise a label. In some embodiments, both a plurality of gNAs and a plurality of nucleic acid-guided nuclease system proteins comprise a label. In some embodiments, the gNAs are targeted a plurality of targets. In some embodiments, the target is a pathogen. In some embodiments, the pathogen is selected from the pathogens in Table 1. In some embodiments, the gNAs targeted to different targets comprise different labels. In some embodiments, the gNAs targeted to different targets comprise the same label. In some embodiments, the nucleic acid-guided nuclease system proteins comprise different labels. In some embodiments, the nucleic acid-guided nuclease system proteins comprise the same label. In some embodiments, the label is a detectable label. In some embodiments, the label is selected from the group consisting of an enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group, an aptamer, one member of a binding pair, and combinations thereof. In some embodiments, the complexes comprise more than one label. In some embodiments, the complexes are attached to a substrate. In some embodiments, the gNAs are attached to the substrate. In some embodiments, the nucleic acid-guided nuclease system proteins are attached to the substrate. In some embodiments, the substrate is silica, plastic, glass, or metal. In some embodiments, the substrate is a 2-dimensional substrate. In some embodiments, the substrate is a 3-dimensional substrate. In some embodiments, the substrate comprises a chamber or is a cylindrical array. In some embodiments, the complexes are attached to a substrate in a known order. In some embodiments, the substrate is reusable. In some embodiments, the complexes are in solution. In some embodiments, the collection comprises at least 1 unique gNA. In some embodiments, the nucleic acid-guided nuclease system protein is a CRISPR/Cas system protein. In some embodiments, the CRISPR/Cas system protein is selected from the group consisting of Cas9, CasX, CasY, Cpf1, Cas3, Cas8a-c, Cas10, Cse1, Csy1, Csn2, Cas4, Csm2, and Cm5. In some embodiments, the nucleic acid-guided nuclease system protein is a dead nucleic acid-guided nuclease system protein. In some embodiments, the CRISPR/Cas system protein is a dead CRISPR/Cas system protein. In some embodiments, the CRISPR/Cas system protein is dCas9. In some embodiments, the nucleic acid-guided nuclease system protein is a nucleic acid-guided nuclease system nickase protein. In some embodiments, the CRISPR/Cas system protein is a CRISPR/Cas system nickase protein. In some embodiments, the nucleic acid-guided nuclease system protein exhibits reduced off-target binding.

In another aspect, provided herein are kits comprising anyone of the collections and compositions described herein.

### BRIEF DESCRIPTION OF THE DRAWINGS

FIG. 1A illustrates exemplary labeling schemes, including a first scheme (left) where the gNAs are labeled and a second scheme (right) where both the gNAs and the nucleic acid-guided nuclease system proteins are labeled. In this figure, the nucleic acid is attached to a substrate.

FIG. 1B illustrates an exemplary scheme for target-specific gNA and nucleic acid-guided nuclease system protein-mediated target detection and identification, where sample nucleic acid is attached to a substrate, and labeled gNA-nucleic acid-guided nuclease system protein complexes are flowed over the substrate. After washing off unbound gNA-nucleic acid-guided nuclease system protein complexes, the target can be identified.

FIG. 2 illustrates an exemplary scheme for target-specific gNA and nucleic acid-guided nuclease system protein-mediated multiplex target detection and identification, where the labeled gNA-nucleic acid-guided nuclease system protein complexes are in solution.

FIG. 3 illustrates an exemplary scheme for target-specific gNA and nucleic acid-guided nuclease system protein-mediated target detection and identification, where the gNA-nucleic acid-guided nuclease system protein complexes are attached to a substrate.

FIGS. 4A-4D illustrate exemplary schemes for target-specific gNA and nucleic acid-guided nuclease system protein-mediated target detection and identification, using a capillary array-based format.

FIG. 4A shows the target-specific gNA-nucleic acid-guided nuclease system protein complexes patterned inside a capillary in segments, in a pre-determined manner, appearing as blocks. Nucleic acids are flowed through the capillary. The detection of signal from the nucleic acid-complex binding within particular portion of the capillary indicates the presence of a target of interest.

FIG. 4B and FIG. 4C illustrate an exemplary scheme where labeled target-specific gNA-nucleic acid-guided nuclease system protein complexes are pre-incubated with sample nucleic acid and then flowed through a capillary array, which also contains target-specific gNA-nucleic acid-guided nuclease system protein complexes patterned in specified manner appearing as blocks inside the capillary. This scheme allows for the more sensitive detection and identification of targets. In this scheme, nucleic acid is associated with two different complexes and is captured.

FIG. 4D shows that target can be detected in a barcode-like manner. The band patterns of the detected signal from nucleic acid-gNA-nucleic acid-guided nuclease system protein complexes can serve as a “UPC barcode” to indicate identities of targets within a biological sample. For example, different E. coli strains within a sample can be identified according to the band pattern.

FIG. 5 illustrates an exemplary scheme for target-specific gNA and nucleic acid-guided nuclease system protein-mediated target identification, using a droplet-based approach.

FIG. 6 illustrates an exemplary scheme for utilizing rolling circle amplification for target detection.

FIG. 7 illustrates an exemplary scheme for utilizing FRET (fluorescence resonance energy transfer) to detect the target.

FIG. 8 illustrates another exemplary scheme for utilizing FRET (fluorescence resonance energy transfer) to detect the target nucleic acid.

FIG. 9 illustrates an exemplary scheme for the fluorescent labeling and detection of target DNA using a nucleic acid-guided nuclease system nickase protein.

FIG. 10 illustrates an exemplary scheme for FRET-mediated detection of target nucleic acid using a nucleic acid-guided nuclease system nickase protein.

### DETAILED DESCRIPTION

Provided herein are methods and compositions for the detection and identification of targets (e.g. pathogens), using target-specific (e.g. pathogen-specific) guide nucleic acids (gNAs) such as gRNAs and nucleic acid-guided nuclease system proteins, such as CRISPR/Cas system proteins. In some preferred embodiments, the gNAs are gRNAs and the nucleic acid-guided nuclease system proteins are CRISPR/Cas system proteins, such as Cas9. Whenever methods employing gRNAs and CRISPR/Cas system proteins are discussed herein, related methods employing other appropriate nucleic acid-guided nuclease system proteins and gNAs are also contemplated.

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Numeric ranges are inclusive of the numbers defining the range.

For purposes of interpreting this specification, the following definitions will apply and whenever appropriate, terms used in the singular will also include the plural and vice versa. If any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

The term “nucleic acid”, as used herein, refers to a molecule comprising one or more nucleic acid subunits. A nucleic acid can include one or more subunits selected from adenosine (A), cytosine (C), guanine (G), thymine (T) and uracil (U), and modified versions of the same. A nucleic acid comprises deoxyribonucleic acid (DNA), ribonucleic acid (RNA), combinations, or derivatives thereof. A nucleic acid may be single-stranded and/or double-stranded.

The nucleic acids comprise “nucleotides”, which, as used herein, is intended to include those moieties that contain purine and pyrimidine bases, and modified versions of the same. Such modifications include methylated purines or pyrimidines, acylated purines or pyrimidines, alkylated riboses or other heterocycles. In addition, the term “nucleotide” or “polynucleotide” includes those moieties that contain hapten or fluorescent labels and may contain not only conventional ribose and deoxyribose sugars, but other sugars as well. Modified nucleosides, nucleotides or polynucleotides also include modifications on the sugar moiety, e.g., wherein one or more of the hydroxyl groups are replaced with halogen atoms or aliphatic groups, or are functionalized as ethers, amines, or the like.

For any of the structural and functional characteristics described herein, methods of determining these characteristics are known in the art.

### Targets

Provided here in are methods and compositions for the detection and identification of targets. The methods utilize target-specific guide nucleic acids (gNAs) (such as guide RNAs) and nucleic acid-guided nuclease system proteins (e.g., CRISPR/Cas system proteins such as Cas9). The target contains a nucleic acid, which is utilized for identification and detection.

Contemplated targets include, but are not limited to, species or genus-level detection in a sample (e.g., the presence of human DNA); pathogens (such as those used in illustrative embodiments below); single nucleotide polymorphisms (SNPs), insertions, deletions, tandem repeats, or translocations (e.g., for tumor profiling or diagnosis of genetic disease); identifying markers indicative of an individual (e.g., a human individual), such as human SNPs or STRs (e.g., for identification of an individual's DNA in a forensic sample); potential toxins; or animals, fungi, and plants (e.g. ricin containing traces of castor plant; raw materials used in food).

In some cases, the genomes of one or more subjects present in a complex sample are substantially identical and can be difficult to resolve using standard technologies. In some cases, the one or more subjects have genomes that are 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999% identical. In some cases, the one or more subjects are one or more different strains of microorganisms, for example, one or more strains of bacteria, virus, fungus and the like.

Target nucleic acid sequences can comprise one or more genetic features. The one or more genetic features can distinguish one subject from another. A genetic feature as referred to herein can be a genome, a genotype, a haplotype, chromatin, a chromosome, a chromosome locus, chromosomal material, an allele, a gene, a gene cluster, a gene locus, a genetic polymorphism, a genetic mutation, a single nucleotide polymorphism (SNP), a restriction fragment length polymorphism (RFLP), a variable tandem repeat (VTR), a copy number variant (CNV), a microsatellite sequence, a genetic marker, a sequence marker, a sequence tagged site (STS), a plasmid, a transcription unit, a transcription product, a gene expression level, a genetic expression state. A target nucleic acid sequence can comprise essentially any known genetic feature.

### Pathogen Targets

Provided here in are methods and compositions for the detection and identification of pathogens. Pathogens can be bacterial, viral, fungal, algal, or protozoal. Pathogens can be eukaryotic or prokaryotic.

In some embodiments, the pathogen is bacterial. In one embodiment, the pathogen is a gram-negative bacteria. In another embodiment, the pathogen is a gram-positive bacteria. In one embodiment the pathogen is a tuberculosis-causing bacteria. In an exemplary embodiment, the pathogen is Mycobacterium tuberculosis. In another embodiment, the pathogen is a bacteria of the genus Escherichia. In another embodiment, the pathogen is an Escherichia coli (E. coli) bacteria. In an exemplary embodiment, the pathogen is E. coli O157:H7. In an exemplary embodiment, the pathogen is E. coli K12. In an exemplary embodiment, the pathogen is E. coli S88. In an exemplary embodiment, the pathogen is E. coli O45:K1.

In some embodiments, the pathogen is a disease-causing pathogen. In some embodiments, the pathogen causes a foodborne infection. In some embodiments, the pathogen causes a urinary tract infection. In some embodiments, the pathogen causes pneumonia. In some embodiments, the pathogen causes an upper respiratory infection. In some embodiments, the pathogen causes sepsis or septic shock. In some embodiments, the pathogen causes a gastrointestinal illness. In some embodiments, the pathogen is a sexually-transmitted pathogen.

In some embodiments, the pathogen is used as a bioweapon. In some embodiments, such a pathogen is anthrax. In one exemplary embodiment, the pathogen is Bacillus anthraces. In one exemplary embodiment, the pathogen is Yersinia pestis.

In some embodiments, the pathogen is a eukaryotic pathogen (is pathogenic in a eukaryotic organisms).

In some embodiments, the pathogen is a mammalian pathogen (can be pathogenic in a mammalian organism). In some embodiments, the pathogen is a human pathogen. In some embodiments, the pathogen is a primate pathogen. In some embodiments, the pathogen is a non-primate pathogen. In some embodiments, the pathogen is a monkey pathogen. In some embodiments, the pathogen is specific for livestock, for example for a horse, a sheep, a cow, a pig, or a donkey. In some embodiments, the pathogen is specific for a domestic animal, for example a cat, a dog, a gerbil, a mouse, or a rat.

In some embodiments, the pathogen is a mammalian parasite. In one embodiment, the parasite is a worm. In another embodiment, the parasite is a malaria-causing parasite. In another embodiment, the parasite is a Leishmaniasis-causing parasite. In another embodiment, the parasite is an amoeba.

In some embodiments, the pathogen is a non-mammalian pathogen (can be pathogenic in a non-mammalian organism).

In some embodiments, the pathogen is a plant pathogen. In some embodiments, the plant is rice, maize, wheat, rose, grape, coffee, fruit, tomato, potato, or cotton.

In some embodiments, the pathogen is an avian pathogen (is pathogenic in birds and other avian organisms). An avian organism includes, but is not limited to, chicken, turkey, duck and goose.

In exemplary embodiments, Table 1 (adapted from the CDC's “Summary of Notifiable Diseases—United States, 2010” accessed on Feb. 18, 2016 at http://www.cdc.gov/mmwr/preview/mmwrhtml/mm5953a1.htm) provides exemplary disease causing pathogens that can be identified using the methods and compositions provided herein.

TABLE 1
Anthrax pathogens
Arboviral pathogens, neuroinvasive and nonneuroinvasive
California serogroup virus
Eastern equine encephalitis virus
Powassan virus
St. Louis encephalitis virus
West Nile virus
Western equine encephalitis virus
Botulism pathogens
Foodborne pathogens
Brucellosis† pathogens
Chancroid
Cholera
Cryptosporidiosis
Cyclosporiasis
Dengue Virus Infection
Dengue fever
Dengue hemorrhagic fever
Dengue shock syndrome
Diphtheria
Ehrlichiosis/Anaplasmosis
Giardiasis
Gonorrhea
Haemophilus influenzae, invasive disease
Hansen disease (leprosy)
Hantavirus pulmonary syndrome
Hemolytic uremic syndrome, post-diarrheal
Hepatitis, viral
Hepatitis A, acute
Hepatitis B, acute
Hepatitis B virus, perinatal infection
Hepatitis B, chronic
Hepatitis C, acute
Hepatitis C, chronic
Human Immunodeficiency Virus (HIV)
Influenza-associated pediatric mortality
Legionellosis
Listeriosis
Lyme disease
Malaria
Measles
Meningococcal disease
Mumps
Novel influenza A virus infections
Pertussis
Plague
Poliomyelitis, paralytic
Poliovirus infection, nonparalytic
Psittacosis
Q fever
Rabies
Rubella
Rubella, congenital syndrome
Salmonellosis
Severe acute respiratory syndrome-associated coronavirus (SARS-CoV)
disease
Shiga toxin-producing (STEC)
Shigellosis
Smallpox
Spotted fever rickettsiosis
Streptococcal toxic-shock syndrome
Streptococcus pneumoniae, invasive disease
Syphilis
Syphilis, congenital
Tetanus
Toxic-shock syndrome (other than streptococcal)
Trichinellosis
Tuberculosis
Tularemia
Typhoid fever
Vancomycin-intermediate (VISA) infection
Vancomycin-resistant (VRSA) infection
Varicella
Vibriosis
Viral hemorrhagic fevers
New World Arenavirus
Crimean-Congo hemorrhagic fever virus
Ebola virus
Lassa virus
Marburg virus
Yellow fever
Zika virus

Target-Specific gNAs

Provided herein are target-specific guide nucleic acids (gNAs). These target-specific gNAs are utilized for the detection and identification of nucleic acid targets in a sample. The gNAs guide the nucleic acid-guided nuclease proteins to the gNA's cognate target, for binding.

In some embodiments, the target-specific gNAs are capable of being labeled.

Also provided herein is a collection of gNAs targeted to at least one target. In some embodiments, the target is a pathogen. In some embodiments, the target is SNP.

In some embodiments, a collection of gNAs is targeted to a single target.

In some embodiments, a collection of gNAs is targeted to a plurality of targets.

In some embodiments, a collection comprises gNAs targeted to at least 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 75, 80, 90, 100, 200, 250, 300, 400, 500, 600, 700, 750, 800, 900, 1000, 2500, 5000, 7500, or even at least 10,000 targets. In some exemplary embodiments, a collection comprises gNAs targeted to about 1-3, 1-5, 1-10, 1-25, 1-50, 1-75, 1-100, 5-10, 5-25, 5-50, 5-75, 5-100, 10-20, 10-25, 10-50, 10-75, 10-100, 25-50, 25-75, 25-100, 50-75, 50-100, 75-100, 100-150, 100-200, 100-300, 100-400, 100-500, 100-600, 100-700, 100-800, 100-900, 100-1000, 100-200 targets, 200-300, 200-400, 200-500, 200-600, 200-700, 200-800, 200-900, 200-1000, 300-400, 300-500, 300-600, 300-700, 300-800, 300-900, 300-1000, 400-500, 400-600, 400-700, 400-800, 400-900, 400-1000, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, 900-1000, 500-1000, 500-5000, 500-10,000, 1000-5000, 1000-10,000, or even about 5000-10,000 targets.

In some embodiments, the target-specific gNAs comprise a label.

In some embodiments, a collection of gNAs is targeted a plurality of targets, and the gNAs targeted to each individual target comprises a different label or gNAs targeted to each individual target comprises multiple labels.

In some embodiments, the gNAs in a collection comprise targeting sequences directed to target sequences spaced every 10⁶ bp, 10⁵ bp, 10⁴ bp, 10³ bp, 10² bp, 50 bp, 25 bp or less across the genome of a target organism.

In some embodiments, the gNAs in a collection comprise targeting sequences directed to unique sequences in the genome of a target organism.

In some embodiments, the gNAs are attached to a substrate. In some embodiments the gNAs are attached to the substrate in a known, referenceable, or predetermined order.

In some embodiments the gNAs are in solution.

In some embodiments, the gNAs are complexed with a nucleic acid-guided nuclease system protein. In some embodiments a collection of gNAs as provided herein comprises members that exhibit specificity to at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or even at least 20 nucleic acid-guided nuclease system proteins. In one specific embodiment, a collection of gNAs as provided herein comprises members that exhibit specificity for a dCas9 protein and another dead Cas/CRISPR system protein selected from the group consisting of deadCpf1, deadCas3, deadCas8a-c, deadCas10, deadCse1, deadCsy1, deadCsn2, deadCas4, deadCsm2, and deadCm5.

In some embodiments, a collection of gNAs targeted at least one target comprises at least 1, at least 5, at least 10, at least 25, at least 50, at least 75, at least 100, at least 250, at least 500, at least 750, at least 1000, at least 2500, at least 5000, at least 75000, at least 10,000, at least 25,000, at least 50,000, at least 100,000, at least 250,000, at least 500,000, at least 750,000, at least 1,000,000, at least 2,500,000, at least 5,000,000, at least 7,500,000, at least 10,000,000 unique gNAs. In some embodiments, a collection of gNAs targeted to at least one target comprises at least 5-10, 10-50, 50-100, 10-100, 100-500, 100-1000, 100-10,000, 100-100,000, 100-1,000,000, 100-10,000,000, 1000-10,000, 1000-100,000, 1000-1,000,000, 1000-10,000,000, 10,000-100,000, 10,000-1,000,000, 10,000-10,000,000, 100,000-1,000,000, 100,000-10,000,000, or 1,000,000-10,000,000 unique gNAs. In some embodiments a collection of gNAs comprise at least 10², at least 10³, at least 10⁴, at least 10⁵, at least 10⁶, at least 10⁷, at least 10⁸, at least 10⁹, at least 10¹⁰ unique gNAs. In some embodiments a collection of gNAs contains a total of at least 10², at least 10³, at least 10⁴, at least 10⁵, at least 10⁶, at least 10⁷, at least 10⁸, at least 10⁹, at least 10¹⁰ gNAs.

In some embodiments, the gNAs comprise any purines or pyrimidines (and/or modified versions of the same). In some embodiments, the gNAs comprise adenine, uracil, guanine, and cytosine (and/or modified versions of the same). In some embodiments, the gNAs comprise adenine, thymine, guanine, and cytosine (and/or modified versions of the same). In some embodiments, the gNAs comprise adenine, thymine, guanine, cytosine and uracil (and/or modified versions of the same).

In some embodiments, the target-specific gNA comprises a first RNA component comprising a targeting sequence and a second RNA component comprising a nucleic acid-guided nuclease system protein-binding sequence. In some embodiments, the first component comprises a crRNA and the second component comprises tracrRNA. In some embodiments, the two components are covalently bound. In some embodiments, the two components are not covalently bound. In some embodiments, the two components are associated with each other.

Pathogen-Specific gNAs

In some embodiments, a collection of gNAs is targeted to a single pathogen, at least one pathogen, or to a plurality of pathogens. In some embodiments, the pathogen causes one or more of the diseases listed in Table 1.

In some embodiments, a collection comprises gNAs targeted to at least 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 75, 80, 90, 100, 200, 250, 300, 400, 500, 600, 700, 750, 800, 900, 1000, 2500, 5000, 7500, or even at least 10,000 pathogens. In some exemplary embodiments, a collection comprises gNAs targeted to about 1-3, 1-5, 1-10, 1-25, 1-50, 1-75, 1-100, 5-10, 5-25, 5-50, 5-75, 5-100, 10-20, 10-25, 10-50, 10-75, 10-100, 25-50, 25-75, 25-100, 50-75, 50-100, 75-100, 100-150, 100-200, 100-300, 100-400, 100-500, 100-600, 100-700, 100-800, 100-900, 100-1000, 100-200 targets, 200-300, 200-400, 200-500, 200-600, 200-700, 200-800, 200-900, 200-1000, 300-400, 300-500, 300-600, 300-700, 300-800, 300-900, 300-1000, 400-500, 400-600, 400-700, 400-800, 400-900, 400-1000, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, 900-1000, 500-1000, 500-5000, 500-10,000, 1000-5000, 1000-10,000, or even about 5000-10,000 pathogens.

In some embodiments, a collection of gNAs is targeted to a plurality of pathogens of the same genus. In some embodiments, a collection of gNAs is targeted to a plurality of pathogens of a different genus.

In some embodiments, a collection of gNAs is targeted to a plurality of pathogens of the same species. In some embodiments, a collection of gNAs is targeted a plurality of pathogens of different species.

In some embodiments, a collection of gNAs is targeted to a plurality of pathogens of the same serotype. In some embodiments, a collection of gNAs is targeted a plurality of pathogens of the differing serotypes.

In some embodiments, the pathogen-specific gNAs comprise a label.

In some embodiments, a collection of gNAs is targeted a plurality of pathogens, and the gNAs targeted to each individual pathogen comprises a different label or gNAs targeted to each individual pathogen comprises multiple labels.

In an exemplary embodiment, a collection of gNAs is targeted to five pathogens, wherein the pathogens are Ebola, HIV, Dengue, Zika, and Chikungunya. In this exemplary collection, the gNAs specific for the Ebola comprise a first label; the gNAs specific for the HIV comprise a second label; the gNAs specific for the Dengue comprise a third label; the gNAs specific for the Zika comprise a fourth label; and the pathogen-specific gNAs specific for the Chikungunya comprise a fifth label.

In some embodiments, the gNAs in a collection comprise targeting sequences directed to pathogen sequences spaced every 10⁶ bp, 10⁵ bp, 10⁴ bp, 10³ bp, 10² bp, 50 bp, 25 bp or less across the genome of a pathogen.

In some embodiments, the pathogen-specific gNAs in a collection comprise targeting sequences directed to unique sequences in the genome of a pathogen.

In some embodiments, the pathogen-specific gNAs are attached to a substrate. In some embodiments the pathogen-specific gNAs are attached to the substrate in a known, referenceable, or predetermined order.

In some embodiments the pathogen-specific gNAs are in solution.

In some embodiments, the pathogen-specific gNAs are complexed with a nucleic acid-guided nuclease system protein. In some embodiments a collection of pathogen-specific gNAs as provided herein comprises members that exhibit specificity to at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or even at least 20 nucleic acid-guided nuclease system proteins. In one specific embodiment, a collection of pathogen-specific gNAs as provided herein comprises members that exhibit specificity for a dCas9 protein and another dead Cas/CRISPR system protein selected from the group consisting of deadCpf1, deadCas3, deadCas8a-c, deadCas10, deadCse1, deadCsy1, deadCsn2, deadCas4, deadCsm2, and deadCm5.

In some embodiments, a collection of pathogen-specific gNAs targeted at least one pathogen comprises at least 1, at least 5, at least 10, at least 25, at least 50, at least 75, at least 100, at least 250, at least 500, at least 750, at least 1000, at least 2500, at least 5000, at least 75000, at least 10,000, at least 25,000, at least 50,000, at least 100,000, at least 250,000, at least 500,000, at least 750,000, at least 1,000,000, at least 2,500,000, at least 5,000,000, at least 7,500,000, at least 10,000,000 unique pathogen-specific gNAs. In some embodiments, a collection of pathogen-specific gNAs targeted to at least one pathogen comprises at least 5-10, 10-50, 50-100, 10-100, 100-500, 100-1000, 100-10,000, 100-100,000, 100-1,000,000, 100-10,000,000, 1000-10,000, 1000-100,000, 1000-1,000,000, 1000-10,000,000, 10,000-100,000, 10,000-1,000,000, 10,000-10,000,000, 100,000-1,000,000, 100,000-10,000,000, or 1,000,000-10,000,000 unique pathogen-specific gNAs. In some embodiments a collection of gNAs comprise at least 10², at least 10³, at least 10⁴, at least 10⁵, at least 10⁶, at least 10⁷, at least 10⁸, at least 10⁹, at least 10¹⁰ unique gNAs. In some embodiments a collection of gNAs contains a total of at least 10², at least 10³, at least 10⁴, at least 10⁵, at least 10⁶, at least 10⁷, at least 10⁸, at least 10⁹, at least 10¹⁰ pathogen-specific gNAs.

In some embodiments, the pathogen-specific gNAs comprise any purines or pyrimidines (and/or modified versions of the same). In some embodiments, the gNAs comprise adenine, uracil, guanine, and cytosine (and/or modified versions of the same). In some embodiments, the gNAs comprise adenine, thymine, guanine, and cytosine (and/or modified versions of the same). In some embodiments, the gNAs comprise adenine, thymine, guanine, cytosine and uracil (and/or modified versions of the same).

In some embodiments the pathogen-specific gNA comprises a first RNA component comprising a targeting sequence and a second RNA component comprising a nucleic acid-guided nuclease system protein-binding sequence. In some embodiments, the first component comprises a crRNA and the second component comprises tracrRNA. In some embodiments the two components are covalently bound. In some embodiments, the two components are not covalently bound. In some embodiments, the two components are associated with each other.

Organization of Target-Specific gNAs

The target-specific gNAs provided herein can be organized in regions on a surface. For example, the target-specific gNAs can be organized as spots, blocks, beads, droplets, wells, or other organization structures on an array or other surface or substrate (e.g., beads, plates). Populations of gNAs can be organized in regions on surface, e.g. in partitions such as wells or droplets. Populations of gNAs can be organized and located with nucleic acid-guided nuclease system proteins to enable binding to targets within the organization.

A given region, whether a spot, block, bead, droplet, well, or other organizational structure, can comprise gNAs targeting a single targeting sequence. In other cases, a given region can comprise a population of guide nucleic acids targeting at least 2, 3, 4, 5, 6, 7, 8, 9, 10 or more targeting sequences. Multiple targeting sequences within a single region can each be different targeting sequences for the same target, such as different targeting sequences that each identify a particular species of pathogen. In other cases, multiple targeting sequences within a single region can each be targeting sequences for different targets, such as different members of the same genus.

Subject-specific regions can be distributed on a surface in such a way as to be individually addressable (e.g., individually addressable for detection), such as in discrete spots or clusters. The plurality of gNAs corresponding to a subject-specific region can be arranged into one or more sets of gNAs. Within each set of gNAs, the plurality of gNAs can be identical or they can be different from one another. Within each set, the plurality of gNAs can each comprise a subject-specific feature. The plurality of gNAs within each set can comprise one or more subject-specific features that distinguish one subject from another. In some cases, a subject-specific feature can be a spot or an area on an array, such as a circular, square, or rectangular area. In some cases, a subject-specific feature can be a bead. In some cases, a subject-specific feature can be a series of gNAs labeled with a feature specific tag. The feature specific tag can be, for example, a feature specific barcode or a binding site for a feature specific label. In some instances, features have replicate features. In some instances, the replicate features are identical. In some instances, the replicate features are designed to identify the same target polynucleotides. In some instances, the replicate features are designed to identify the same genome. In some instances, the replicate features are designed to identify any strain within a species. In some cases, the replicate features are designed to identify an individual.

Multiple unique gNAs within a gNAs set or subject-specific region can be located in an area that is smaller than or is comparable in size to the resolution of a detection system employed to detect signal from the device. The area encompassed by multiple unique and ordered gNAs could be less than the resolution of the detection system, equal to the resolution of the detection system, or the area encompassed by all the unique gNAs could be larger as long as the area encompassed by at least 2 of the randomly ordered unique gNAs in the set is roughly equivalent to, or less than, the resolution of the detection system. In such cases, signal from multiple unique gNAs or features can be collected or integrated in one or few pixels, or other resolution elements. Such an approach can achieve similar results as pooling non-identical guide nucleic acids into a single feature.

gNAs can be designed to detect false positives. For example, gNAs can be designed by designing gNA sets wherein the individual gNAs within the set are designed with one or more bases that are mismatched to individual guide nucleic acids in other gNA sets. In some cases, the gNA sets are complementary. In other cases, the gNA sets are not complementary. In another example, a gNA set can be designed to search for a subject organism that has multiple similar strains. In this example, a gNA set could be added to detect individual sequences contained in strains that are not target but have genomes that are very close to that of the target with one or more individual unique characteristics.

Regions can each be different including, without limitation, containing a different number of gNAs, different types of gNAs, different subject-specific features targeted by the gNAs, different average representations of unique gNAs, and the like.

### Nucleic Acid-Guided Nuclease System Proteins

Methods of the present disclosure utilize nucleic acid-guided nucleases. As used herein, a “nucleic acid-guided nuclease” is any nuclease that cleaves DNA, RNA or DNA/RNA hybrids, and which uses one or more nucleic acid guide nucleic acids (gNAs) to confer specificity. Nucleic acid-guided nucleases include CRISPR/Cas system proteins as well as non-CRISPR/Cas system proteins.

The nucleic acid-guided nucleases provided herein can be DNA guided DNA nucleases; DNA guided RNA nucleases; RNA guided DNA nucleases; or RNA guided RNA nucleases. The nucleases can be endonucleases. The nucleases can be exonucleases. In one embodiment, the nucleic acid-guided nuclease is a nucleic acid-guided-DNA endonuclease. In one embodiment, the nucleic acid-guided nuclease is a nucleic acid-guided-RNA endonuclease.

A nucleic acid-guided nuclease system protein-binding sequence is a nucleic acid sequence that binds any protein member of a nucleic acid-guided nuclease system. For example, a CRISPR/Cas system protein-binding sequence is a nucleic acid sequence that binds any protein member of a CRISPR/Cas system.

In some embodiments, the nucleic acid-guided nuclease is selected from the group consisting of CAS Class I Type I, CAS Class I Type III, CAS Class I Type IV, CAS Class II Type II, and CAS Class II Type V proteins. In some embodiments, nucleic acid-guided nuclease system proteins comprise CRISPR/Cas system proteins, including proteins from CRISPR Type I systems, CRISPR Type II systems, and CRISPR Type III systems. In some embodiments, the nucleic acid-guided nuclease is selected from the group consisting of Cas9, CasX, CasY, Cpf1, Cas3, Cas8a-c, Cas10, Cse1, Csy1, Csn2, Cas4, Csm2, Cm5, Csf1, C2c2, and NgAgo. In an exemplary embodiment, the nucleic acid-guided nuclease is Cas9.

In some embodiments, nucleic acid-guided nuclease system proteins can be from any bacterial or archaeal species.

In some embodiments, the nucleic acid-guided nuclease system proteins are from, or are derived from nucleic acid-guided nuclease system proteins from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

In some embodiments, the nucleic acid-guided nuclease system proteins are naturally occurring. In some embodiments, the nucleic acid-guided nuclease system proteins are engineered. In some embodiments, the nucleic acid-guided nuclease system proteins are isolated, recombinantly produced, or synthetic.

In some embodiments, naturally occurring nucleic acid-guided nuclease system proteins comprise CRISPR/Cas system proteins including Cas9, CasX, CasY, Cpf1, Cas3, Cas8a-c, Cas10, Cse1, Csy1, Csn2, Cas4, Csm2, and Cm5.

In some embodiments, engineered examples of nucleic acid-guided nuclease system proteins include catalytically dead nucleic acid-guided nuclease system proteins. The term “catalytically dead” or simply “dead nucleic acid-guided nuclease system protein” generally refers to a nucleic acid-guided nuclease system protein that has, for example in the case of some CRISPR/Cas system proteins, inactivated HNH and RuvC nucleases. Such a protein can bind to a target site in any nucleic acid (where the target site is determined by the gNA), but the protein is unable to cleave or nick the double-stranded DNA. In some embodiments, the nucleic acid-guided nuclease system catalytically dead protein is a catalytically dead Cas9 (dCas9). In one embodiment, a dead nucleic acid-guided nuclease system protein-gNA complex binds to targets determined by the gNA sequence. The dead nucleic acid-guided nuclease system protein bound can prevent cutting by nucleic acid-guided nucleases system proteins while other manipulations proceed.

In some embodiments, engineered examples of nucleic acid-guided nuclease system proteins also include nucleic acid-guided nuclease system nickase protein. A nucleic acid-guided nuclease system nickase refers to a modified version of a nucleic acid-guided nuclease system protein, containing a single inactive catalytic domain. In one embodiment, the nucleic acid-guided nuclease system nickase is Cas9 nickase. A Cas9 nickase may contain a single inactive catalytic domain, for example, either the RuvC- or the HNH-domain. With only one active nuclease domain, the nucleic acid-guided nuclease nickase cuts only one strand of the target DNA, creating a single-strand break or “nick”. Depending on which mutant is used, the gNA-hybridized strand or the non-hybridized strand may be cleaved. nucleic acid-guided nuclease nickases bound to 2 gNAs that target opposite strands will create a double-strand break in the DNA. This “dual nickase” strategy can increase the specificity of cutting because it requires that both gNA-nucleic acid-guided nuclease complexes (e.g., Cas9/gRNA complexes) be specifically bound at a site before a double-strand break is formed.

In some embodiments, engineered examples of nucleic acid-guided nuclease system proteins also include a high-specificity mutant of a nucleic acid-guided nuclease system protein, containing amino acid changes that confer reduced off-target binding to similar but not identical sequences to the target.

In some embodiments, engineered examples of nucleic acid-guided nuclease system proteins also include nucleic acid-guided nuclease system fusion proteins. For example, a nucleic acid-guided nuclease system protein may be fused to another protein, for example an activator, a repressor, a nuclease, an enzyme, a fluorescent molecule, a chemical tag, a radioactive tag, or a transposase. For example, the nucleic acid-guided nuclease system protein can be fused to a EGFP, or a terminal transferase.

In some embodiments, the nucleic acid-guided nuclease system proteins are attached to a substrate. In some embodiments, the nucleic acid-guided nuclease system proteins are in solution.

### CRISPR/Cas System Nucleic Acid-Guided Nucleases

In some embodiments, CRISPR/Cas system proteins are used. In some embodiments, CRISPR/Cas system proteins include proteins from CRISPR Type I systems, CRISPR Type II systems, and CRISPR Type III systems.

In some embodiments, CRISPR/Cas system proteins can be from any bacterial or archaeal species.

In some embodiments, the CRISPR/Cas system protein is isolated, recombinantly produced, or synthetic. In some embodiments, examples of CRISPR/Cas system proteins can be naturally occurring, can mimic naturally occurring versions or, can be engineered versions.

In some embodiments, the CRISPR/Cas system proteins are from, or are derived from CRISPR/Cas system proteins from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

In some embodiments, naturally occurring CRISPR/Cas system proteins can belong to CAS Class I Type I, III, or IV, or CAS Class II Type II or V, and can include Cas9, Cas3, Cas8a-c, Cas10, Cse1, Csy1, Csn2, Cas4, Csm2, Cmr5, Csf1, C2c2, and Cpf1.

In an exemplary embodiment, the CRISPR/Cas system protein comprises Cas9.

A “CRISPR/Cas system protein-gNA complex” refers to a complex comprising a CRISPR/Cas system protein and a gNA (e.g. a gRNA or a gDNA). Where the gNA is a gRNA, the gRNA may be composed of two molecules, i.e., one RNA (“crRNA”) which hybridizes to a target and provides sequence specificity, and one RNA, the “tracrRNA”, which is capable of hybridizing to the crRNA. Alternatively, the guide RNA may be a single molecule (i.e., a gRNA) that contains crRNA and tracrRNA sequences.

A CRISPR/Cas system protein may be at least 60% identical (e.g., at least 70%, at least 80%, or 90% identical, at least 95% identical or at least 98% identical or at least 99% identical) to a wild type CRISPR/Cas system protein. The CRISPR/Cas system protein may have all the functions of a wild type CRISPR/Cas system protein, or only one or some of the functions, including binding activity, nuclease activity, and nuclease activity.

The term “CRISPR/Cas system protein-associated gNA” refers to a gNA. The CRISPR/Cas system protein-associated gNA may exist as isolated NA, or as part of a CRISPR/Cas system protein-gNA complex.

### Cas9

In some embodiments, the CRISPR/Cas System protein nucleic acid-guided nuclease is Cas9 or comprises Cas9. The Cas9 of the present invention can be isolated, recombinantly produced, or synthetic. The Cas9 can be naturally occurring, can mimic naturally occurring versions or, can be an engineered version.

Examples of Cas9 proteins that can be used in the embodiments herein can be found in F. A. Ran, L. Cong, W. X. Yan, D. A. Scott, J. S. Gootenberg, A. J. Kriz, B. Zetsche, O. Shalem, X. Wu, K. S. Makarova, E. V. Koonin, P. A. Sharp, and F. Zhang; “In vivo genome editing using Staphylococcus aureus Cas9,” Nature 520, 186-191 (9 Apr. 2015) doi:10.1038/nature14299, which is incorporated herein by reference.

In some embodiments, the Cas9 is a Type II CRISPR system derived from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, or Corynebacter diphtheria.

In some embodiments, the Cas9 is a Type II CRISPR system derived from S. pyogenes and the PAM sequence is NGG located on the immediate 3′ end of the target specific guide sequence. The PAM sequences of Type II CRISPR systems from exemplary bacterial species can also include: Streptococcus pyogenes (NGG), Staph aureus (NNGRRT), Neisseria meningitidis (NNNNGA TT), Streptococcus thermophilus (NNAGAA) and Treponema denticola (NAAAAC) which are all usable without deviating from the present invention.

In one exemplary embodiment, Cas9 sequence can be obtained, for example, from the pX330 plasmid (available from Addgene), re-amplified by PCR then cloned into pET30 (from EMD biosciences) to express in bacteria and purify the recombinant 6His tagged protein.

A “Cas9-gNA complex” refers to a complex comprising a Cas9 protein and a gNA. A Cas9 protein may be at least 60% identical (e.g., at least 70%, at least 80%, or 90% identical, at least 95% identical or at least 98% identical or at least 99% identical) to a wild type Cas9 protein, e.g., to the Streptococcus pyogenes Cas9 protein. The Cas9 protein may have all the functions of a wild type Cas9 protein, or only one or some of the functions, including binding activity, nuclease activity, and nuclease activity.

The term “Cas9-associated gNA” refers to a gNA as described above. The Cas9-associated gNA may exist isolated, or as part of a Cas9-gNA complex.

### Non-CRISPR/Cas System Nucleic Acid-Guided Nucleases

In some embodiments, non-CRISPR/Cas system proteins are used in the embodiments provided herein.

In some embodiments, the non-CRISPR/Cas system proteins can be from any bacterial or archaeal species.

In some embodiments, the non-CRISPR/Cas system protein is isolated, recombinantly produced, or synthetic.

In some embodiments, the non-CRISPR/Cas system proteins are from, or are derived from Aquifex aeolicus, Thermus thermophilus, Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis, Natronobacterium gregoryi, or Corynebacter diphtheria.

In some embodiments, the non-CRISPR/Cas system proteins can be naturally occurring, can mimic naturally occurring versions or, can be engineered versions

In some embodiments, a naturally occurring non-CRISPR/Cas system protein is NgAgo (Argonaute from Natronobacterium gregoryi).

A “non-CRISPR/Cas system protein-gNA complex” refers to a complex comprising a non-CRISPR/Cas system protein and a gNA (e.g. a gRNA or a gDNA). Where the gNA is a gRNA, the gRNA may be composed of two molecules, i.e., one RNA (“crRNA”) which hybridizes to a target and provides sequence specificity, and one RNA, the “tracrRNA”, which is capable of hybridizing to the crRNA. Alternatively, the guide RNA may be a single molecule (i.e., a gRNA) that contains crRNA and tracrRNA sequences.

A non-CRISPR/Cas system protein may be at least 60% identical (e.g., at least 70%, at least 80%, or 90% identical, at least 95% identical or at least 98% identical or at least 99% identical) to a wild type non-CRISPR/Cas system protein. The non-CRISPR/Cas system protein may have all the functions of a wild type non-CRISPR/Cas system protein, or only one or some of the functions, including binding activity, nuclease activity, and nuclease activity.

The term “non-CRISPR/Cas system protein-associated gNA” refers to a gNA. The non-CRISPR/Cas system protein-associated gNA may exist as isolated NA, or as part of a non-CRISPR/Cas system protein-gNA complex.

### Catalytically Dead Nucleic Acid-Guided Nucleases

In some embodiments, engineered examples of nucleic acid-guided nucleases include catalytically dead nucleic acid-guided nucleases (CRISPR/Cas system nucleic acid-guided nucleases or non-CRISPR/Cas system nucleic acid-guided nucleases). The term “catalytically dead” generally refers to a nucleic acid-guided nuclease that has inactivated nucleases, for example inactivated HNH and RuvC nucleases. Such a protein can bind to a target site in any nucleic acid (where the target site is determined by the gNA), but the protein is unable to cleave or nick the nucleic acid.

Accordingly, the catalytically dead nucleic acid-guided nuclease allows separation of the mixture into unbound nucleic acids and catalytically dead nucleic acid-guided nuclease-bound fragments. In one exemplary embodiment, a dCas9/gRNA complex binds to the targets determined by the gRNA sequence. The dCas9 bound can prevent cutting by Cas9 while other manipulations proceed.

In another embodiment, the catalytically dead nucleic acid-guided nuclease can be fused to another enzyme, such as a transposase, to target that enzyme's activity to a specific site.

In some embodiments, the catalytically dead nucleic acid-guided nuclease is dCas9, dCpf1, dCas3, dCas8a-c, dCas10, dCse1, dCsy1, dCsn2, dCas4, dCsm2, dCm5, dCsf1, dC2C2, or dNgAgo.

In one exemplary embodiment, the catalytically dead nucleic acid-guided nuclease protein is a dCas9.

### Nucleic Acid-Guided Nuclease Nickases

In some embodiments, engineered examples of nucleic acid-guided nucleases include nucleic acid-guided nuclease nickases (referred to interchangeably as nickase nucleic acid-guided nucleases).

In some embodiments, engineered examples of nucleic acid-guided nucleases include CRISPR/Cas system nickases or non-CRISPR/Cas system nickases, containing a single inactive catalytic domain.

In some embodiments, the nucleic acid-guided nuclease nickase is a Cas9 nickase, Cpf1 nickase, Cas3 nickase, Cas8a-c nickase, Cas10 nickase, Cse1 nickase, Csy1 nickase, Csn2 nickase, Cas4 nickase, Csm2 nickase, Cm5 nickase, Csf1 nickase, C2C2 nickase, or a NgAgo nickase.

In one embodiment, the nucleic acid-guided nuclease nickase is a Cas9 nickase.

In some embodiments, a nucleic acid-guided nuclease nickase can be used to bind to target sequence. With only one active nuclease domain, the nucleic acid-guided nuclease nickase cuts only one strand of a target DNA, creating a single-strand break or “nick”. Depending on which mutant is used, the gNA-hybridized strand or the non-hybridized strand may be cleaved. nucleic acid-guided nuclease nickases bound to 2 gNAs that target opposite strands can create a double-strand break in the nucleic acid. This “dual nickase” strategy increases the specificity of cutting because it requires that both nucleic acid-guided nuclease/gNA complexes be specifically bound at a site before a double-strand break is formed.

In exemplary embodiments, a Cas9 nickase can be used to bind to target sequence. The term “Cas9 nickase” refers to a modified version of the Cas9 protein, containing a single inactive catalytic domain, i.e., either the RuvC- or the HNH-domain. With only one active nuclease domain, the Cas9 nickase cuts only one strand of the target DNA, creating a single-strand break or “nick”. Depending on which mutant is used, the guide RNA-hybridized strand or the non-hybridized strand may be cleaved. Cas9 nickases bound to 2 gRNAs that target opposite strands will create a double-strand break in the DNA. This “dual nickase” strategy can increase the specificity of cutting because it requires that both Cas9/gRNA complexes be specifically bound at a site before a double-strand break is formed.

Capture of DNA can be carried out using a nucleic acid-guided nuclease nickase. In one exemplary embodiment, a nucleic acid-guided nuclease nickase cuts a single strand of double stranded nucleic acid, wherein the double stranded region comprises methylated nucleotides.

### Dissociable and Thermostable Nucleic Acid-Guided Nucleases

In some embodiments, thermostable nucleic acid-guided nucleases are used in the methods provided herein (thermostable CRISPR/Cas system nucleic acid-guided nucleases or thermostable non-CRISPR/Cas system nucleic acid-guided nucleases). In such embodiments, the reaction temperature is elevated, inducing dissociation of the protein; the reaction temperature is lowered, allowing for the generation of additional cleaved target sequences. In some embodiments, thermostable nucleic acid-guided nucleases maintain at least 50% activity, at least 55% activity, at least 60% activity, at least 65% activity, at least 70% activity, at least 75% activity, at least 80% activity, at least 85% activity, at least 90% activity, at least 95% activity, at least 96% activity, at least 97% activity, at least 98% activity, at least 99% activity, or 100% activity, when maintained for at least 75° C. for at least 1 minute. In some embodiments, thermostable nucleic acid-guided nucleases maintain at least 50% activity, when maintained for at least 1 minute at least at 75° C., at least at 80° C., at least at 85° C., at least at 90° C., at least at 91° C., at least at 92° C., at least at 93° C., at least at 94° C., at least at 95° C., 96° C., at least at 97° C., at least at 98° C., at least at 99° C., or at least at 100° C. In some embodiments, thermostable nucleic acid-guided nucleases maintain at least 50% activity, when maintained at least at 75° C. for at least 1 minute, 2 minutes, 3 minutes, 4 minutes, or 5 minutes. In some embodiments, a thermostable nucleic acid-guided nuclease maintains at least 50% activity when the temperature is elevated, lowered to 25° C.−50° C. In some embodiments, the temperature is lowered to 25° C., to 30° C., to 35° C., to 40° C., to 45° C., or to 50° C. In one exemplary embodiment, a thermostable enzyme retains at least 90% activity after 1 min at 95° C.

In some embodiments, the thermostable nucleic acid-guided nuclease is thermostable Cas9, thermostable Cpf1, thermostable Cas3, thermostable Cas8a-c, thermostable Cas10, thermostable Cse1, thermostable Csy1, thermostable Csn2, thermostable Cas4, thermostable Csm2, thermostable Cm5, thermostable Csf1, thermostable C2C2, or thermostable NgAgo.

In some embodiments, the thermostable CRISPR/Cas system protein is thermostable Cas9.

Thermostable nucleic acid-guided nucleases can be isolated, for example, identified by sequence homology in the genome of thermophilic bacteria Streptococcus thermophilus and Pyrococcus furiosus. Nucleic acid-guided nuclease genes can then be cloned into an expression vector. In one exemplary embodiment, a thermostable Cas9 protein is isolated.

In another embodiment, a thermostable nucleic acid-guided nuclease can be obtained by in vitro evolution of a non-thermostable nucleic acid-guided nuclease. The sequence of a nucleic acid-guided nuclease can be mutagenized to improve its thermostability.

Complexes of Target-Specific gNAs and Nucleic Acid-Guided Nuclease System Proteins

Provided herein are nucleic acid-guided nuclease system proteins that are complexed to target specific guide nucleic acids (gNAs). The gNAs direct the nucleic acid-guided nuclease system proteins to the target nucleic acid. A nucleic acid-guided nuclease system can be an RNA-guided nuclease system. A nucleic acid-guided nuclease system can be a DNA-guided nuclease system. In some cases, the nucleic acid-guided nuclease system proteins are nucleic acid-guided nuclease system proteins that are complexed to target-specific gNAs and direct the nucleic acid-guided nuclease system protein to a RNA target. In some cases, the nucleic acid-guided nuclease system proteins are nucleic acid-guided nuclease system proteins that are complexed to target-specific gNAs and direct the nucleic acid-guided nuclease system protein to a DNA target.

Provided herein are target-specific gNAs complexed to nucleic acid-guided nuclease system proteins. Also provided herein are collections of gNA-nucleic acid-guided nuclease system protein complexes, wherein the gNAs are targeted to at least one target (e.g. pathogen-specific gNAs).

In some embodiments, the complexes comprise a label. In some embodiments, the label is a detectable label.

In some embodiments, the gNAs in the collection of complexes are targeted to a plurality of targets.

In some embodiments, the complexes comprise more than one label.

In some embodiments, the complexes are attached to a substrate. In some embodiments, the complexes are attached to a substrate in a known or pre-determined order. In some embodiments, the substrate is reusable.

In some embodiments, the complexes are in solution.

### Samples

Provided herein methods and compositions for the detection and identification of a target in a sample, based on the nucleic acid present in the sample.

The contemplated samples include, but are not limited to biological samples, clinical samples, forensic samples, environmental samples, metagenomic samples, and the like.

In some embodiments, the sample is a tumor tissue sample.

In some embodiments, the sample is blood, serum, plasma mucus, hair, urine, feces, saliva, breath, cerebrospinal fluid, lymph, tissue, skin, or a biopsy.

In some embodiments, the sample is a food sample, for example a sample of meat, dairy, or produce.

In some embodiments, the sample is a fabric sample.

In some embodiments, the sample is a soil sample. In some embodiments, the sample is a rock sample. In some embodiments, the sample is plant sample.

In some embodiments, the sample is a water sample.

In some embodiments the sample is an air sample. In some embodiments the sample is derived from an air filter.

In some embodiments the sample is a processed sample. In some embodiments, the sample is an unprocessed sample.

In some embodiments, the sample comprises DNA.

In some embodiments, the sample comprises RNA. In some embodiments, the sample comprises RNA and is reverse transcribed to produce cDNA.

In some embodiments, the nucleic acid in the sample is sheared prior to use. In some embodiments, the nucleic acid in the sample is enzymatically sheared. In some embodiments, the nucleic acid in the sample is mechanically sheared.

In some embodiments, the nucleic acid in the sample (e.g. the DNA) is amplified prior to use.

In some embodiments, the nucleic acid in the sample (e.g. the DNA) is circularized prior to use.

In some embodiments, the nucleic acid in the sample (e.g. the DNA) is amplified by rolling circle amplification (RCA).

In some embodiments, the nucleic acid in the sample (e.g. the DNA) is amplified by an isothermal amplification technique, including but not limited to loop-mediated isothermal amplification (LAMP), helicase-dependent amplification, nicking enzyme amplification reaction (NEAR), and recombinase polymerase amplification (RPA).

Generally the size of the target nucleic acid to be identified ranges from 20 bp-10⁹ bp.

In some embodiments, the nucleic acid in the sample (e.g. the DNA) is blocked with dideoxy CTP (a non-extendable nucleotide). In some embodiments, the nucleic acid in the sample (e.g. the DNA) is blocked at one or more termini with non-extendible nucleotides.

### Labels

Provided herein are methods and compositions for the detection and identification of targets, using target-specific gNAs and nucleic acid-guided nuclease system proteins. The method comprises contacting a nucleic acid in the sample from a sample with target-specific gNA-nucleic acid-guided nuclease system protein complexes. In some embodiments the nucleic acid comprises, or is, DNA. In some embodiments, the nucleic acid comprises, or is RNA.

In some embodiments, the target-specific gNAs comprise a label, or are capable of being labeled. Merely by way of example, fluorescent RNA-binding dyes include, but are not limited to, SYBR Green II, OliGreen, and RiboGreen.

In some embodiments, the nucleic acid-guided nuclease system proteins comprise a label, or are capable of being labeled (e.g., comprise a reactive site to which a label can be attached).

In some embodiments, the target-specific gNA-nucleic acid-guided nuclease system protein complexes comprise a label, or are capable of being labeled. In some embodiments, the target-specific gNAs are labeled prior to contacting nucleic acid and the nucleic acid-guided nuclease system protein is labeled after contacting the nucleic acid. In some embodiments, the target-specific gNAs are labeled after contacting the nucleic acid and the nucleic acid-guided nuclease system protein is labeled prior to contacting the nucleic acid. In some embodiments, both the target-specific gNAs and the nucleic acid-guided nuclease system protein are labeled prior to contacting the nucleic acid. In some embodiments, both the target-specific gNAs and the nucleic acid-guided nuclease system protein are labeled after contacting the nucleic acid.

In some embodiments, the target-specific gNAs and the nucleic acid comprise a label, or are capable of being labeled. In some embodiments, the nucleic acid-guided nuclease system proteins and the nucleic acid comprise a label, or are capable of being labeled.

In some embodiments, the target-specific gNA-nucleic acid-guided nuclease system protein complexes and the nucleic acid comprise a label, or are capable of being labeled.

In some embodiments, the same label is used to tag different targets that belong to the same group. For example, in some embodiments, all gNAs specific for a bacterial pathogen comprise with a first label, all gNAs specific for viral pathogens comprise a second label, and all gNAs specific for fungal pathogens comprise a third label.

In some embodiments, different labels are used to tag different pathogens that belong to the same group. For example, gNAs specific for E. coli bacteria comprise a first label, and gNAs specific for B. subtilis bacteria comprise a second label.

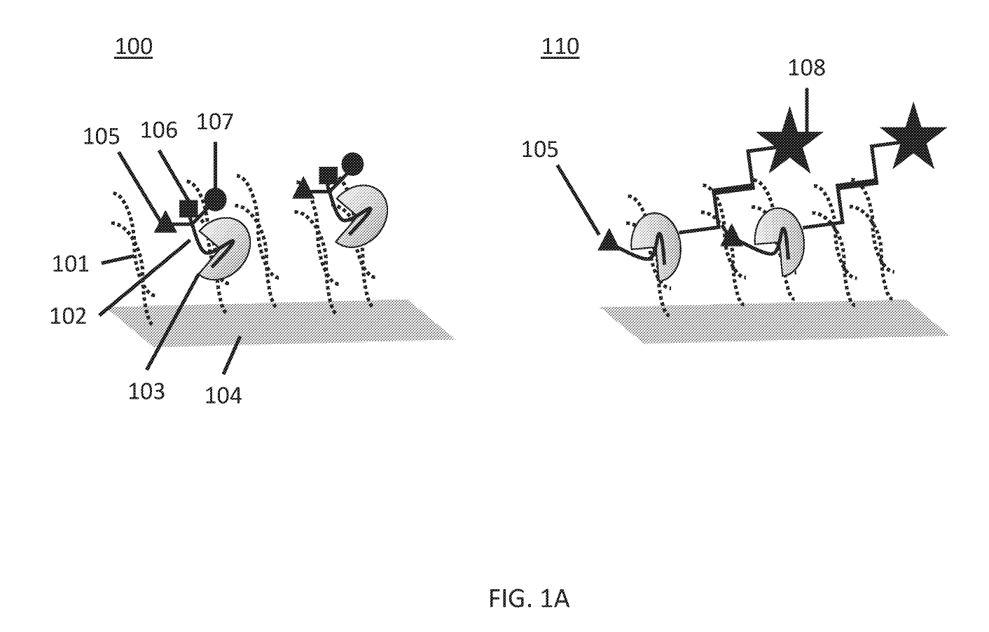

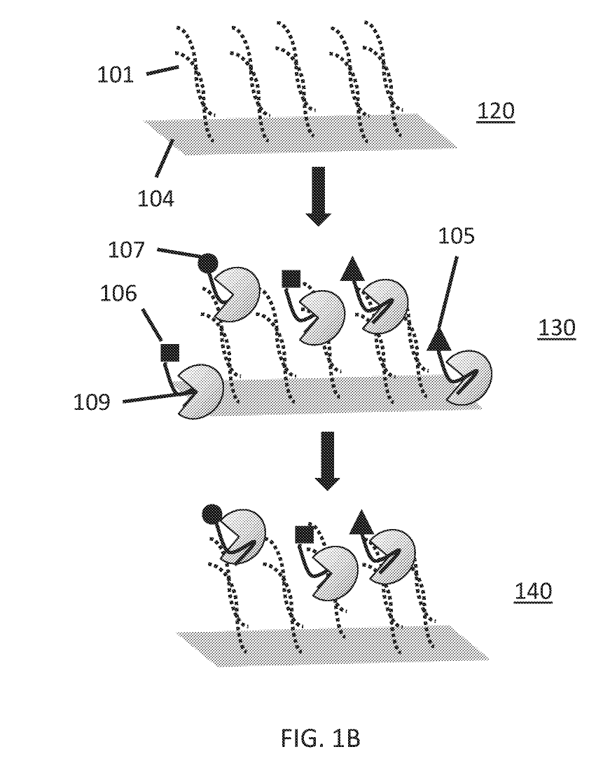

In some embodiments, a complex comprising target-specific gNA 102 and a nucleic acid-guided nuclease system protein 103 is labeled with multiple labels or multi-component labels (see, e.g., FIG. 1A and FIG. 1B), such as multiple fluorophores, multiple biochemical molecules (e.g. horseradish peroxidase, HRP), or combinations thereof. In some cases, the target (e.g., sample DNA 101) can be bound to a substrate 104. In some embodiments, the multiple labels can be used to trigger a signal amplification cascade. In some embodiments, the target-specific gNA is labeled with multi-component 105 106 107 tags (see, e.g., FIG. 1A 100). In some embodiments, both the target-specific gNA and the nucleic acid-guided nuclease system proteins are labeled with multi-component labels 105 108 (see, e.g., FIG. 1A 110). In some embodiments, the nucleic acid-guided nuclease system protein is labeled with multi-component tags (see, e.g., FIG. 1B).

In some embodiments, target-specific gNA and the nucleic acid-guided nuclease system proteins are both labeled with the same labels.

In some embodiments, target-specific gNAs and the nucleic acid-guided nuclease system proteins are labeled with different labels. In some embodiments, detection and localization of both labels, for example on a substrate, indicates that a complex has been formed between the gNA and the nucleic acid-guided nuclease system protein.

In some embodiments, complexes targeting different targets comprise different labels. In some embodiments, complexes targeting different targets comprise the same label, but are attached to a substrate in a known order. In some embodiments, complexes targeting different target do not comprise a label.

In some embodiments, the nucleic acid comprises a label, or is capable of being labeled.

In some embodiments, the nucleic acid label is an intercalating label.

In some embodiments, the nucleic acid label is a non-specific nucleic acid-binding label.

In some embodiments, the nucleic acid comprises a nucleic acid dye label. Examples of suitable light-emitting nucleic acid dyes include, but are not limited to, EvaGreen dye, GelRed, GelGreen, SYBR Green I (U.S. Pat. Nos. 5,436,134 and 5,658,751), SYBR GreenEr, SYBR Gold, LC Green, LC Green Plus, BOXTO, BEBO, SYBR DX, SYTO9, SYTOX Blue, SYTOX Green, SYTOX Orange, SYTO dyes, POPO-1, POPO-3, BOBO-1, BOBO-3, YOYO-1, YOYO-3, TOTO-1, TOTO-3, PO-PRO-1, BO-PRO-1, YO-PRO-1, TO-PRO-1, JO-PRO-1, PO-PRO-3, LO-PRO-1, BO-PRO-3, YO-PRO-3, TO-PRO-3, TO-PRO-5, Ethidium Homodimer-1, Ethidium Homodimer-2, Ethidium Homodimer-3, propidium iodide, ethidium bromide, various Hoechst dyes, 4′,6-diamidino-2-phenylindole (DAPI), ResoLight, Chromofy, and acridine homodimer Other nucleic acid dyes include those disclosed in U.S. Pat. No. 4,883,867 to Lee (1989), U.S. Pat. No. 5,582,977 to Yue et al. (1996), U.S. Pat. No. 5,321,130 to Yue et al. (1994), U.S. Pat. No. 5,410,030 to Yue et al. (1995), U.S. Pat. No. 5,863,753, and U.S. Patent Publication Nos. 2006/0211028 and 2008/0145526. Many of the above mentioned dyes are commercially available from Invitrogen, Sigma, Biotium and numerous other companies.

In some embodiments, the nucleic acid is labeled prior to being contacted with the target-specific guide NA-nucleic acid-guided nuclease system protein complexes. In some embodiments, the nucleic acid is labeled after being contacted with the target-specific guide NA-nucleic acid-guided nuclease system protein complexes.

In some embodiments, the target-specific gNA-nucleic acid-guided nuclease system protein complexes are labeled prior to being contacted with the nucleic acid. In some embodiments, the target-specific gNA-nucleic acid-guided nuclease system protein complexes are labeled after being contacted with the nucleic acid.

In some embodiments, both the nucleic acid and the target-specific gNA-nucleic acid-guided nuclease system protein complexes are labeled prior to the contacting of the nucleic acid to the complexes. In some embodiments, both the nucleic acid and the target-specific gNA-nucleic acid-guided nuclease system protein complexes are labeled after the contacting of the nucleic acid to the complexes.

In some embodiments, different methods are utilized to detect the label on the nucleic acid and the label on the gNA-nucleic acid-guided nuclease system protein complex.

In some embodiments, the nucleic acid and the gNA-nucleic acid-guided nuclease system protein complex are labeled for signal detection based upon principles of fluorescent resonance energy transfer (FRET). For example, nucleic acid can be labeled with YOYO-1 intercalator dye (donor) and the gNA-nucleic acid-guided nuclease system protein complex can be labeled with Cy3 (acceptor). When the nucleic acid is bound by the gNA-nucleic acid-guided nuclease system protein complex (and a donor/acceptor pair is created), a sensitized Cy3 emission will be detectable. Exemplary donor moieties include, but are not limited to, YOYO-1, Cy5, Cy3, DY-630, DiD, Dy-635, and exemplary acceptor moieties include, but are not limited to, Alexa Fluor® dyes such as Alexa Fluor® 647, Alexa Fluor® 350, Alexa Fluor® 405, and Alexa Fluor® 430.

In some embodiments, the label is a moiety that is further capable of being attached to a label.

In some embodiments, the label is a detectable label.

Contemplated labels include, but are not limited to an enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group (capable of undergoing a redox reaction), an aptamer, one member of a binding pair, and combinations thereof.

In specific embodiments, the label is a fluorophore. A fluorophore can be any substance which absorbs light of one wavelength and emits light of a different wavelength. Typical fluorophores include fluorescent dyes, semiconductor nanocrystals, lanthanide chelates, and fluorescent proteins. Exemplary fluorescent dyes include fluorescein, 6-FAM, rhodamine, Texas Red, tetramethylrhodamine, a carboxyrhodamine, carboxyrhodamine 6G, carboxyrhodol, carboxyrhodamine 110, Cascade Blue, Cascade Yellow, coumarin, Cy2 ®, Cy3 ®, Cy3.5 ®, Cy5 ®, Cy5.5 ®, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor® 350, Alexa Fluor® 430, Alexa Fluor® 488, Alexa Fluor® 532, Alexa Fluor® 546, Alexa Fluor® 568, Alexa Fluor® 594, Alexa Fluor® 633, Alexa Fluor® 647, Alexa Fluor® 660, Alexa Fluor® 680, 7-amino-4-methylcoumarin-3-acetic acid, BODIPY FL, BODIPY FL-Br2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, Quantum Dots, conjugates thereof, and combinations thereof.

Exemplary lanthanide chelates include europium chelates, terbium chelates and samarium chelates.

Exemplary enzymes include alkaline phosphatase, horseradish peroxidase, beta-galactosidase, glucose oxidase, galactose oxidase, neuraminidase, a bacterial luciferase, an insect luciferase and sea pansy luciferase (Renilla koellikeri), which can create a detectable signal in the presence of suitable substrates and assay conditions, known in the art.

Exemplary haptens and/or members of a binding pair include avidin, streptavidin, digoxigenin, biotin, and those described above.

In some embodiments, the nucleic acid, the target-specific gNAs, the nucleic acid-guided nuclease system proteins, or the gNA-nucleic acid-guided nuclease complexes are labeled with different labels.

In some embodiments, detection and localization of labels, for example on a substrate, indicates that a complex has been formed between the gNA and the nucleic acid-guided nuclease system protein. In one exemplary embodiment, the target-specific gNA and the nucleic acid-guided nuclease system protein are labeled for signal detection based upon principles of fluorescent resonance energy transfer (FRET). In such an embodiment, one label is donor moiety, and the other label is acceptor moiety. For example, the nucleic acid-guided nuclease system protein label is detectable unless quenched by signal from the target-specific gNA label (or vice versa), when the target-specific gNA and nucleic acid-guided nuclease system protein form a complex (donor/acceptor pair). Another FRET pair is a gNA comprising a donor moiety; and a gNA comprising an acceptor moiety.

Another FRET pair is a gNA comprising a donor moiety; and a nucleic acid (e.g. a DNA) comprising an acceptor moiety.

Another FRET pair is a gNA comprising an acceptor moiety; and a nucleic acid (e.g. a DNA) comprising a donor moiety.

Another FRET pair is a nucleic acid-guided nuclease system protein comprising a donor moiety; and a nucleic acid-guided nuclease system protein comprising an acceptor moiety.

Another FRET pair is a nucleic acid-guided nuclease system protein comprising a donor moiety; and a nucleic acid (e.g. a DNA)comprising an acceptor moiety.

Another FRET pair is a nucleic acid (e.g. a DNA)comprising a donor moiety; and a nucleic acid-guided nuclease system protein comprising an acceptor moiety.

In some embodiments, only the gNAs or the only the nucleic acid-guided nuclease system proteins comprise FRET labels.

For FRET embodiments, exemplary donor moieties include, but are not limited to, YOYO-1, Cy5, Cy3, DY-630, DiD, Dy-635, and exemplary acceptor moieties include, but are not limited to, Alexa Fluor® dyes such as Alexa Fluor® 647, Alexa Fluor® 350, Alexa Fluor® 405, and Alexa Fluor® 430.

Detection

Detection of the labels of the invention can be carried out with standard methods known in the art. For example, detection can be achieved by eye, by detecting a visual color change, by using a spectrophotometer, a fluorescence reader, or a fluorescent microscope. In some embodiments, a hand held unit is utilized for detection. In some embodiments, signal detection is based upon principles of fluorescent resonance energy transfer (FRET), a FRET signal can be detected using a FRET channel on a microscope.

In some embodiments, detection can be performed in multiple steps. In some embodiments, detection is performed on multiple detecting systems.

In an exemplary embodiment, a two-step detection is performed. In this embodiment, a label in the solution is initially detected (e.g. a color change), indicating a further detection step is required on the sample.

Substrates

The current invention provides methods and compositions for detecting and identifying a target using target-specific gNAs and nucleic acid-guided nuclease system proteins. Provided herein are a variety of substrates useful for this purpose.

In some embodiments, target-specific gNAs are attached to a substrate. In some embodiments, the target-specific gNAs are attached to a substrate in a known/pre-determined/referenceable order.

In some embodiments, nucleic acid-guided nuclease system proteins are attached to a substrate.

In some embodiments complexes comprising target-specific gNAs and nucleic acid-guided nuclease system protein complexes are attached to a substrate.

In some embodiments, the nucleic acid to be identified to identify the target is attached to a substrate. In some of the embodiments provided herein, the target nucleic acid comprises, or is, DNA. In some of the embodiments provided herein, the target nucleic acid comprises, or is, RNA.

A gNA, nucleic acid, nucleic acid-guided nuclease system protein, or a complex comprising the same, is attached to a substrate when it is associated with the substrate through a non-random chemical or physical interaction. In some embodiments, the attachment is through a covalent bond. In some embodiments, the nucleic acid is reversibly bound to carboxyl molecules on the surface of the substrate.

The substrate can be made of glass, plastic, silicon, silica-based materials, functionalized polystyrene, functionalized polyethyleneglycol, functionalized organic polymers, nitrocellulose or nylon membranes, paper, cotton, and materials suitable for synthesis. Substrates need not be flat.

In some embodiments, the substrate is a 2-dimensional array. In some embodiments, the 2-dimensional array is flat. In some embodiments, the 2-dimensional array is not flat, for example, the array is a wave-like array.

Substrates include any type of shape including spherical shapes (e.g., beads). Materials attached to substrates may be attached to any portion of the substrates (e.g., may be attached to an interior portion of a porous substrates material).

In some embodiments, the substrate is a 3-dimensional array, for example, a microsphere.

In some embodiments, the microsphere is magnetic. In some embodiments, the microsphere is glass. In some embodiments, the microsphere is made of polystyrene. In some embodiments, the microsphere is silica-based.

In some embodiments, the substrate is an array with interior surface, for example, is a straw, tube, capillary, cylindrical, or microfluidic chamber array. In some embodiments, the substrate comprises multiple straws, capillaries, tubes, cylinders, or chambers. In some embodiments, the outer surface of the substrate is tethered with sample nucleic acid or target-specific gNA-nucleic acid-guided nuclease system protein complexes. In some embodiments, the interior surface of the substrate is tethered with sample nucleic acid or target-specific gNA-nucleic acid-guided nuclease system protein complexes. In some embodiments, both the outer surface and the interior surfaces of the substrate is tethered with sample nucleic acid or target-specific gNA-nucleic acid-guided nuclease system protein complexes.

A gNA, nucleic acid, nucleic acid-guided nuclease system protein, or a complex can be attached to a substrate via a linker. A linker is a chemical moiety that is attachable to a substrate on one end and the gNA, nucleic acid, nucleic acid-guided nuclease system protein, or complex on the other end. The linker comprise atoms or molecules that link or bond two entities, but that is not a part of either of the individual linked entities. In general, linker molecules are oligomeric chain moieties containing 1-500 linearly connected chemical bonds. In some embodiments, the linker contains PEG linkers, I-Linker™ (Integrated DNA Technologies) modifiers, amino modifiers, thiol modiers, etc. In some embodiments, photolabile linkers are used to attach gNAs or nucleic acid-guided nuclease system proteins to the substrate surface, and upon light irradiation, the complexes can be released. In some embodiments, the complexes are be released by enzyme digestion or chemical degradation at the site of linkers.

In some embodiments, the complexes are attached to the substrate via the nucleic acid-guided nuclease system proteins, such as using substrates coated with antibodies against nucleic acid-guided nuclease system proteins, or directly immobilizing the nucleic acid-guided nuclease system proteins on the substrate.

In some embodiments, nucleic acids are attached to the substrate in a known order.

In some embodiments, gNAs are in vitro transcribed on substrate and then complexed with nucleic acid-guided nuclease system proteins.

In some embodiments, the substrate is reusable. In such embodiments, the substrate can comprise target-specific gNAs or target-specific gNA-nucleic acid-guided nuclease system protein complexes attached to the substrate in a known order. The gNAs and gNA-nucleic acid-guided nuclease system protein complexes can be organized in spots, blocks, beads, droplets, wells, or other organization structures on a given substrate. In some embodiments, the nucleic acid is stripped off, and the gNA-nucleic acid-guided nuclease system protein complexes remain for reuse.

A substrate can contain gNAs from 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 75, 80, 90, 100, 200, 250, 300, 400, 500, 600, 700, 750, 800, 900, 1000, 2500, 5000, 7500, or even at least 10,000 targets. In some exemplary embodiments, a substrate comprises gNAs targeted to about 1-3, 1-5, 1-10, 1-25, 1-50, 1-75, 1-100, 5-10, 5-25, 5-50, 5-75, 5-100, 10-20, 10-25, 10-50, 10-75, 10-100, 25-50, 25-75, 25-100, 50-75, 50-100, 75-100, 100-150, 100-200, 100-300, 100-400, 100-500, 100-600, 100-700, 100-800, 100-900, 100-1000, 100-200, 200-300, 200-400, 200-500, 200-600, 200-700, 200-800, 200-900, 200-1000, 300-400, 300-500, 300-600, 300-700, 300-800, 300-900, 300-1000, 400-500, 400-600, 400-700, 400-800, 400-900, 400-1000, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, 900-1000, 500-1000, 500-5000, 500-10,000, 1000-5000, 1000-10,000 targets, or even about 5000-10,000 targets.

A substrate can comprise at least 1, at least 5, at least 10, at least 25, at least 50, at least 75, at least 100, at least 250, at least 500, at least 750, at least 1000, at least 2500, at least 5000, at least 75000, at least 10,000, at least 25,000, at least 50,000, at least 100,000, at least 250,000, at least 500,000, at least 750,000, at least 1,000,000, at least 2,500,000, at least 5,000,000, at least 7,500,000, at least 10,000,000 unique gNAs attached to the substrate. In some embodiments, a substrate comprises at least 5-10, 10-50, 50-100, 10-100, 100-500, 100-1000, 100-10,000, 100-100,000, 100-1,000,000, 100-10,000,000, 1000-10,000, 1000-100,000, 1000-1,000,000, 1000-10,000,000, 10,000-100,000, 10,000-1,000,000, 10,000-10,000,000, 100,000-1,000,000, 100,000-10,000,000, or 1,000,000-10,000,000 unique gNAs attached to the substrate. In some embodiments a substrate comprises at least 10², at least 10³, at least 10⁴, at least 10⁵, at least 10⁶, at least 10⁷, at least 10⁸, at least 10⁹, at least 10¹⁰ unique gNAs attached to the substrate.

### Methods of the Invention

Provided herein are methods and for the detection and identification of targets (i.e. target nucleic acids, target DNA and/or target RNA), using target-specific gNAs and nucleic acid-guided nuclease system proteins.

In particular, provided herein is a method of identifying a target in a sample comprising: (a) contacting nucleic acid from a sample with a plurality of gNA-nucleic acid-guided nuclease system protein complexes, wherein the complexes are targeted to at least one target, and wherein the nucleic acid, the gNA-nucleic acid-guided nuclease system protein complexes, or both the nucleic acid and the gNA-nucleic acid-guided nuclease system protein complexes comprise a label; and (b) identifying the target in the sample, wherein the identifying is achieved by detecting a specific signal from the label, wherein the presence of a specific signal indicates binding of a gNA-nucleic acid-guided nuclease system protein complex to the nucleic acid. As provided herein, the nucleic acid of the target can be DNA, RNA, or a mixture of the two.

The methods of the invention can be carried out under any operating conditions. For example the methods can be carried out at 0° C.-100° C. In some embodiments, the method is carried out at 0° C., 25° C., 37° C., 50° C., 72° C., or even 100° C. In an exemplary embodiment, the method is carried out at room temperature. In an exemplary embodiment, the method is carried out at temperature range of 50°-80° C.

The methods of the invention can be carried out in under 10 minutes, in under 15 minutes in under 30 minutes, in under 60 minutes, in under 90 minutes, in under 120 minutes, in under 150 minutes, in under 180 minutes, in under 210 minutes, in under 240 minutes, in under 270 minutes, in under 300 minutes, in under 330 minutes, in under 360 minutes, in under 7 hours, in under 8 hours, in under 9 hours, in under 10 hours, in under 11 hours, in under 12 hours, in under 15 hours, in under 20 hours, in under 24 hours, or in under 36 hours.

Specific embodiments of the methods of the invention are discussed as exemplary schemes in turn below. It is to be noted that the exemplary schemes illustrate an embodiment of the invention where the target nucleic acid comprises DNA. It is to be understood that this is illustrative only, and that the methods and compositions provided herein are applicable for target identification when the target nucleic acid comprises DNA, RNA, or comprises a mixture of DNA and RNA.

As described and provided herein, the target nucleic acid (e.g., DNA), the gNAs (e.g., gRNAs), the nucleic acid-guided nuclease system proteins (e.g., CRISPR/Cas system proteins), the gNA-nucleic acid-guided nuclease system protein complexes, or combinations of the same can comprise a label (e.g., 105, 106, 107, 108). This is illustrated, for example, in FIG. 1A. Panel 100 illustrates an embodiment where the gNAs 102 are labeled. Panel 110 illustrates a scheme where both the gNAs and the nucleic acid-guided nuclease system proteins 103 are labeled. In this figure, the target nucleic acid (e.g. DNA) 101 to be identified is attached to a substrate 104. In some embodiments, here and below, the nucleic acid-guided nuclease system protein can be a mutant catalytically dead nucleic acid-guided nuclease system protein, e.g. dCas9. Use of this mutant can be advantageous because it would bind, but not cut the target DNA, leaving the DNA intact for downstream applications such as sequencing. Catalytically active nucleic acid-guided nuclease system proteins can be employed, as they can remain bound to a portion of the target nucleic acid subsequent to cutting.

FIG. 1B illustrates an exemplary embodiment for target-specific gNA and nucleic acid-guided nuclease system protein-mediated target identification. In this embodiment, target nucleic acid (e.g., DNA) 101 is isolated and purified from a sample of interest and is attached to a substrate 104 (see, e.g., step 120). Labeled gNA-nucleic acid-guided nuclease system protein complexes 109 are then flowed over the substrate (see, e.g., step 130). The gNAs are directed to three targets in this exemplary scheme for illustrative purposes, and each set of gNA specific for one of the targets comprises a different label 105 106 107. After allowing the DNA and the complexes to bind, the unbound complexes are washed off (see, e.g., step 140). The signals are read from the labels, and target(s) are identified. In some embodiments, DNA bound by the complex can be recovered for further analyses. For example, DNA can be stripped off the substrates; and DNA bound to the complexes can be purified using antibodies against nucleic acid-guided nuclease system proteins, recovered by denaturing nucleic acid-guided nuclease system protein, and then subject to downstream analyses such as DNA sequencing.

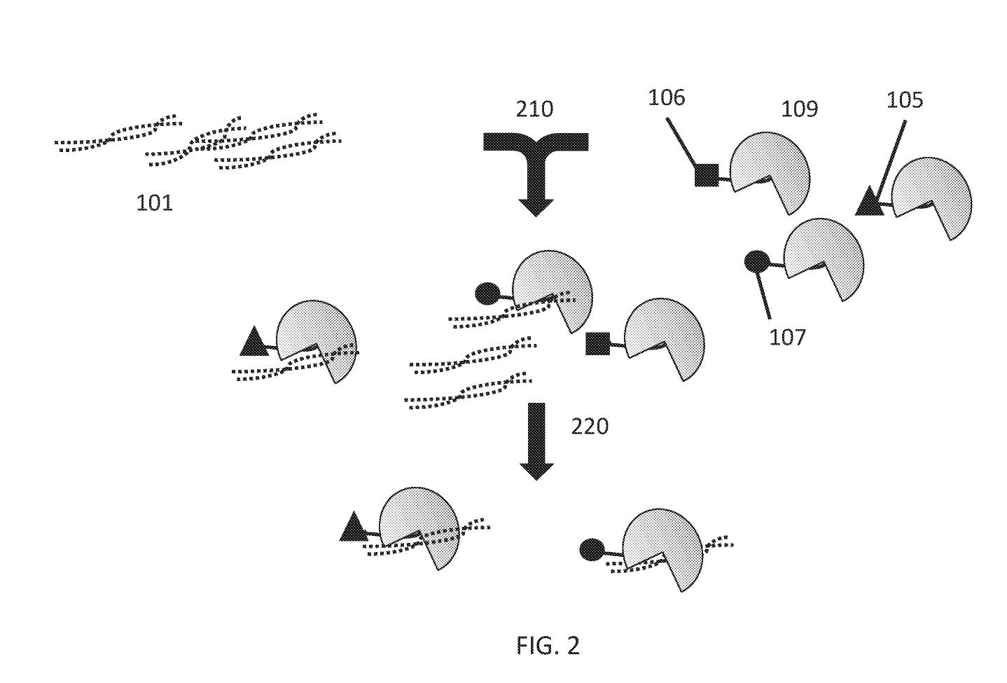

In another exemplary embodiment depicted in FIG. 2, nucleic acids (e.g. DNA) are isolated from a sample of interest, and is optionally purified. The DNA is incubated 210 with a collection of complexes comprising target-specific gNA and nucleic acid-guided nuclease system proteins, in solution, at room temperature. In this embodiment, the target-specific gNAs comprise labels. In this exemplary embodiment picture in FIG. 2, the collection of complexes comprise gNAs specific for at three targets, and comprise three different labels 105 106 107. The complexes bind to the target DNA in the sample. The DNA can optionally be bound to beads before or after adding the complexes. Unbound complexes can be washed away 220 prior to detection. Target DNA within the sample can be detected by the presence of labels. The DNA bound by the complexes can be further recovered for additional downstream analyses. For example, DNA bound to the complexes can be purified using antibodies against nucleic acid-guided nuclease system proteins, recovered by denaturing nucleic acid-guided nuclease system protein, and subject to downstream analyses such as DNA sequencing, SNP analysis, genotyping, or other analyses.

In another exemplary embodiment, the target detection method comprises an initial target screening step. In some embodiments, a sample is obtained and stained with nucleic acid dyes. This will allow for an initial determination of whether any nucleic acid is even present within the sample. In other embodiments, a sample is subjected to an initial screen by incubating the sample with a first detection marker such as HRP-labeled complexes comprising target-specific gNA and nucleic acid-guided nuclease system proteins, at room temperature. Unbound complexes are washed away. Substrate for the HRP is added and further incubated. If the sample contains the target nucleic acids (e.g. DNA), it will change color. Such a rapid initial screening steps can determine which samples require further processing, for more detailed identification. In some embodiments, a further identification of target DNA within the sample is followed. In this multi-screening context, multi-component tags are particularly useful—for example the complex could contain a HRP label for initial detection, and a fluorophore label for more detailed identification.

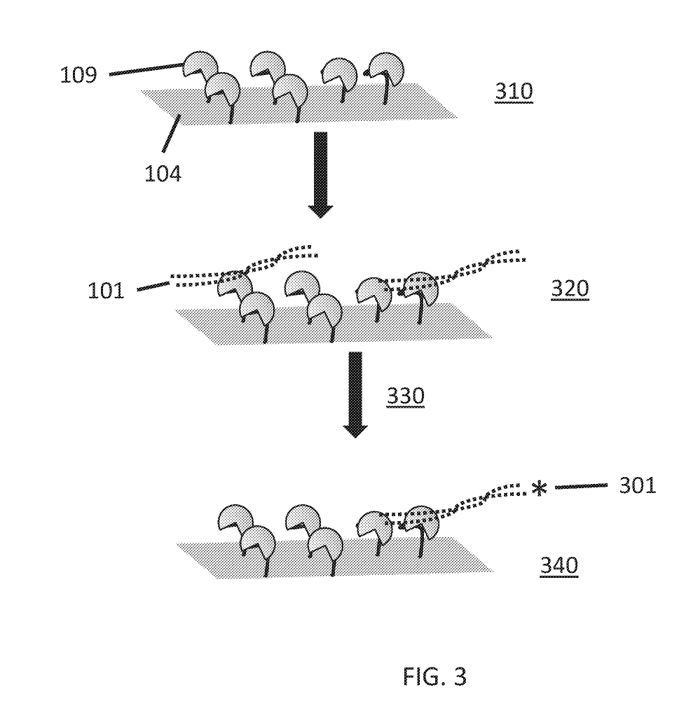

In another exemplary embodiment (FIG. 3), complexes comprising target-specific gNA and nucleic acid-guided nuclease system proteins are attached to a substrate in a known, referenceable, and pre-determined order. Either the gNAs can be attached to the substrate, and then complexed with nucleic acid-guided nuclease system proteins; or the nucleic acid-guided nuclease system proteins can be attached to the substrate, and then complexes with the gNAs. Pictured in FIG. 3 is an embodiment where gNAs are attached to the substrate 104, and then complexed with nucleic acid-guided nuclease system proteins to form complexes 109 (see, e.g., step 310). Nucleic acid (e.g. DNA) 101 isolated from a sample of interest is flowed over the substrate (e.g., at room temperature) (see, e.g., step 320). Unbound DNA is washed off (see, e.g., step 330); and the target DNA bound to the complexes on the substrate is identified according to the position of the detecting signal. In some embodiments, the DNA sample is stained with nucleic acid dye 301 before being flowed over the substrate. In some embodiments, the DNA bound by the complexes is stained with nucleic acid dye 301 (see, e.g., step 340). Since the target-specific gNAs are posited in known positions on the substrate, the position of the signal indicates the identity of the target. In some embodiments, the gNAs, complexes, or nucleic acid-guided nuclease system proteins comprise first fluorophore of a FRET pair; and the DNA is stained with a second fluorophore of a FRET pair. In this embodiment, only upon interaction of the donor/acceptor pairs will a signal be detected. In some embodiments, the target DNA is retrieved and further analyzed. Bound DNA can be subsequently removed from the substrate and further analyzed.

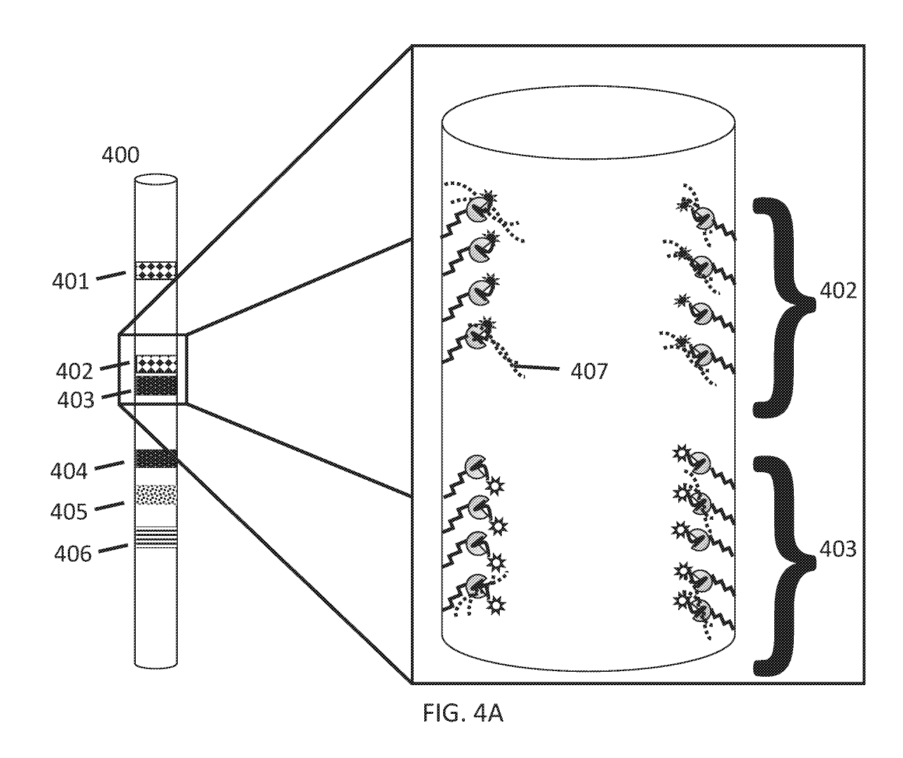

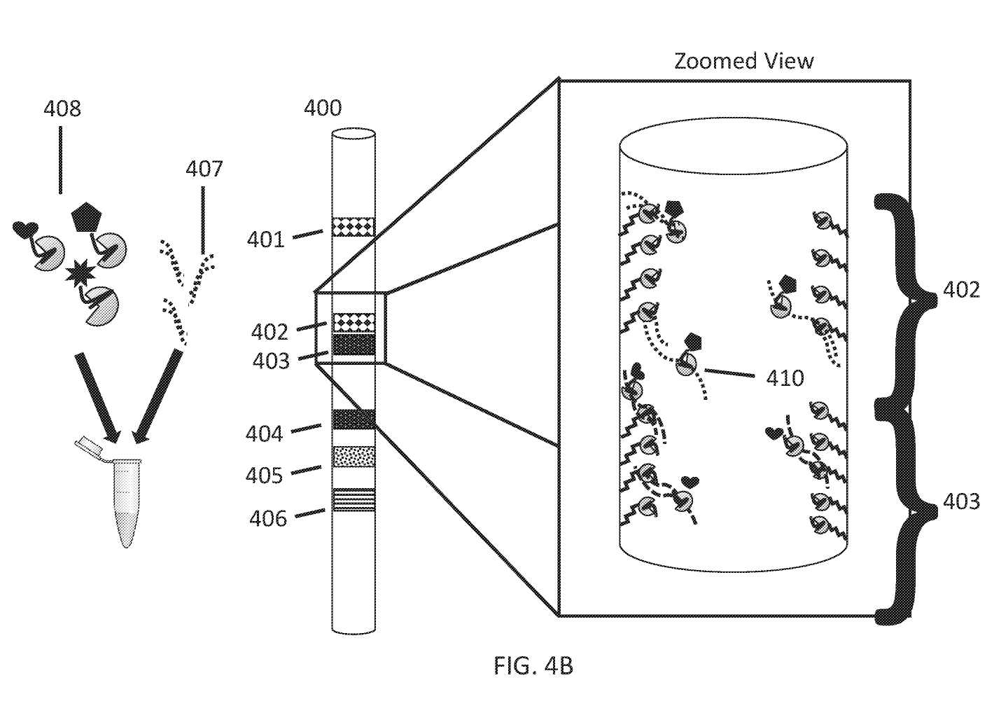

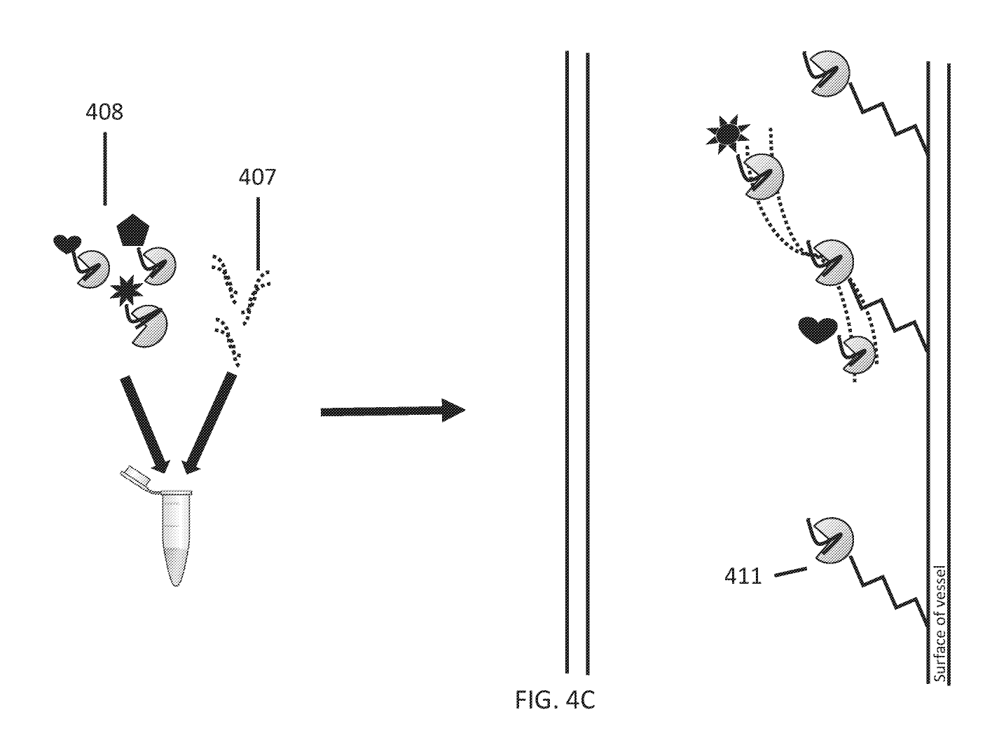

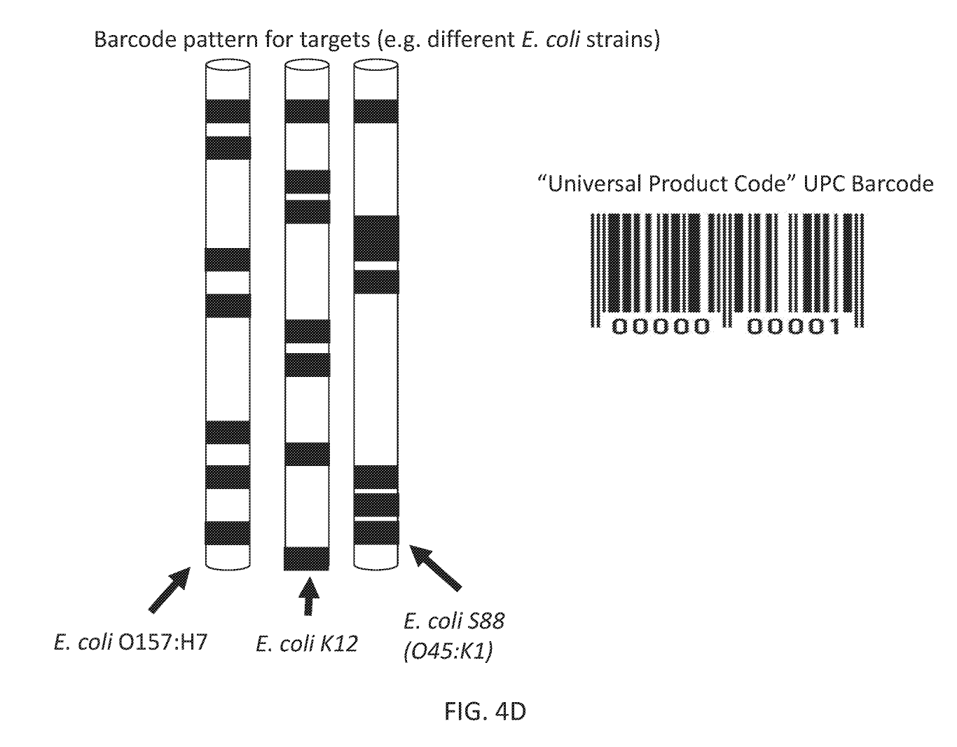

In one particular embodiment, depicted for example in FIGS. 4A-4D, complexes comprising target-specific gNA and nucleic acid-guided nuclease system proteins are tethered inside a capillary, channel, or other flow system 400 (e.g., straw, tube, chamber flow cell, or cylindrical array) in segments with specified pattern appearing as regions or blocks (401 402 403 404 405 406). After flowing isolated and purified nucleic acids (e.g. DNA) 407 from a sample of interest through the capillary and washing off unbound DNA, the appearance of color change along the full length or partial length of the tubing indicates presence of target of interest (see FIG. 4A). In some embodiments, washing off the unbound DNA can be performed in the capillary, for example with an attached pipette bulb. In some embodiments, the isolated and purified DNA is labeled before it is flowed over the capillary. In some embodiments, the isolated and purified DNA is stained with a nucleic acid dye after binding to the complexes tethered inside the capillary.

FIG. 4B and FIG. 4C illustrate an exemplary scheme where target-specific gNA-nucleic acid-guided nuclease system protein complexes 408 are pre-incubated with sample DNA 407 and then passed through a capillary array 400, which also contain target-specific gNA-nucleic acid-guided nuclease system protein complexes patterned on the array in regions or blocks (401 402 403 404 405 406). This scheme allows for a more sensitive detection and identification of target. In this scenario, the gNA-nucleic acid-guided nuclease system 408 is incubated with the sample, and if the target nucleic acid (e.g. DNA) 407 is present in the sample, the gNA-nucleic acid-guided nuclease 408 will bind to it. This sample DNA and bound gNA-nucleic acid-guided nuclease complex 410 will then be allowed to incubate further with the capillary array of covalently linked gNA-nucleic acid-guided nuclease complexes 411 directed at target DNA. These surface-bound gNA-nucleic acid-guided nuclease complexes will then find their targets on any target DNA and bind them, in the process also capturing the initial gNA-nucleic acid-guided nuclease complexes bound to the DNA. In some embodiments, the system can capture large fragments with multiple associated labels. Detection can be read by color change of the whole or in specific parts of the assay format. In this example, the capillary array is shown striped. These stripes can appear as one color to the eye or multiple colors. If the stripes appear as one color to the eye, a more sensitive detection under a spectrometer can be used to examine detailed stripe patterns.

In another embodiment, FIG. 4D shows that the pathogen can be detected in a barcode-type manner. In this scheme, one pattern of blocks or bands indicates one specific subtype or species, and another pattern indicates another subtype or species. For example, one banding pattern or color can indicate pathogen genus, such as Bacillus; other bands can further indicate a narrower definition of the pathogen species, such as a band for Bacillus anthraces, a band for Bacillus cereus, and so forth. Or, for example, one band can indicate the E. coli species (e.g., E. coli O157:H7, E. coli K12, E. coli S88 (O45:K1)), and the remaining bands can further define the strain by different, unique patterns of gNAs (see, e.g., FIG. 4D). This banding pattern can be used to narrow down pathogen identity, similar to a UPC barcode. Bands can also be used that are specific for certain characteristics such as pathogenicity and resistance.

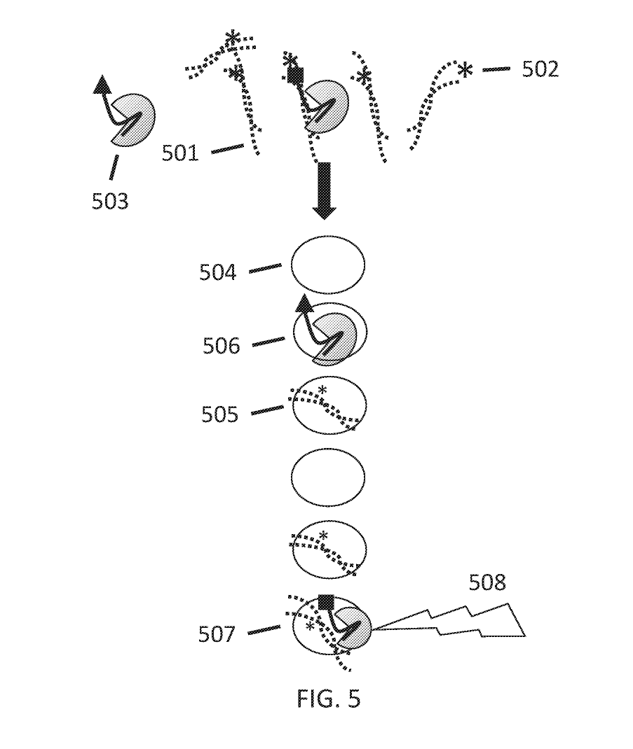

In another exemplary embodiment (FIG. 5), DNA 501 isolated and purified from a sample of interest is stained with nucleic acid dye 502, and incubated with labeled complexes 503 comprising target-specific gNA and nucleic acid-guided nuclease system proteins. The mixture of nucleic acids (e.g. DNA) and complexes are partitioned, for example into droplets or wells. Droplets can be formed by combining the DNA and complex mixture with droplet generation oil, and transferring the fluid to a droplet generator device (e.g., microfluidic cartridge). An emulsion of 10²-10¹⁰ monodispersed, nanoliter-sized droplets can be generated. A given droplet in the population can be empty 504 (in some cases, most droplets obtained are empty), a given droplet can contain one DNA 505, a given droplet can contain one complex 506, or a given droplet can contain one DNA bound by a complex 507. The detection of signals 508 from both labeled complexes and nucleic acid dye-stained DNA indicate the presence of the target DNA of interest. In some embodiments, the procedure is carried out on a QX200™ Droplet Digital™ PCR System (Bio-Rad), Raindrop™ system by Raindance or other pico, nano or micro liter droplet formation systems. In some embodiments, the droplets containing signals from both labeled complexes and nucleic acid dye-stained DNA are recovered, and the bound DNAs are recovered for further downstream analyses, such as sequencing and cloning.

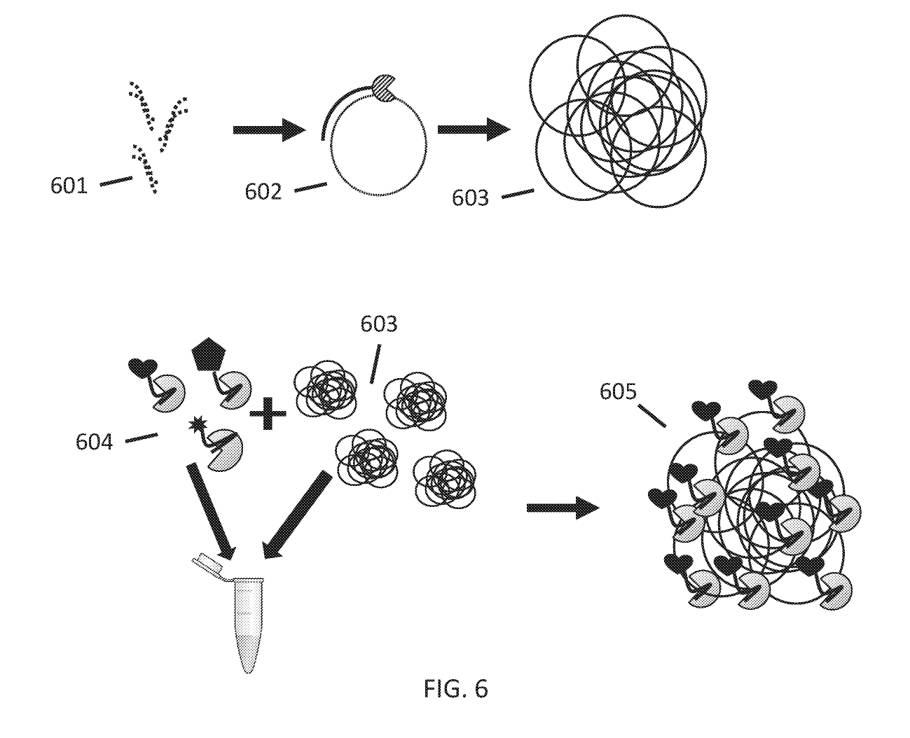

In another exemplary embodiment (see FIG. 6), nucleic acids (e.g. DNA) 601 isolated and purified from a sample of interest are circularized and amplified by Rolling Circle Amplification (RCA) 602. The product of the RCA 603 is then allowed to incubate with complexes comprising target-specific gNA and nucleic acid-guided nuclease system proteins. The gNA directs the labeled nucleic acid-guided nuclease system protein to the multiple copies of the target on the RCA product, thus labeling the RCA product with one label 605. In one embodiment, the complexes are bound to a substrate surface and the RCA product is flowed over the substrate. In another embodiment, the RCA product is attached to a substrate and the complexes are washed over the RCA product. In another embodiment, the detection occurs in solution. Upon binding, the identity of the target is detected, based on the label detected.

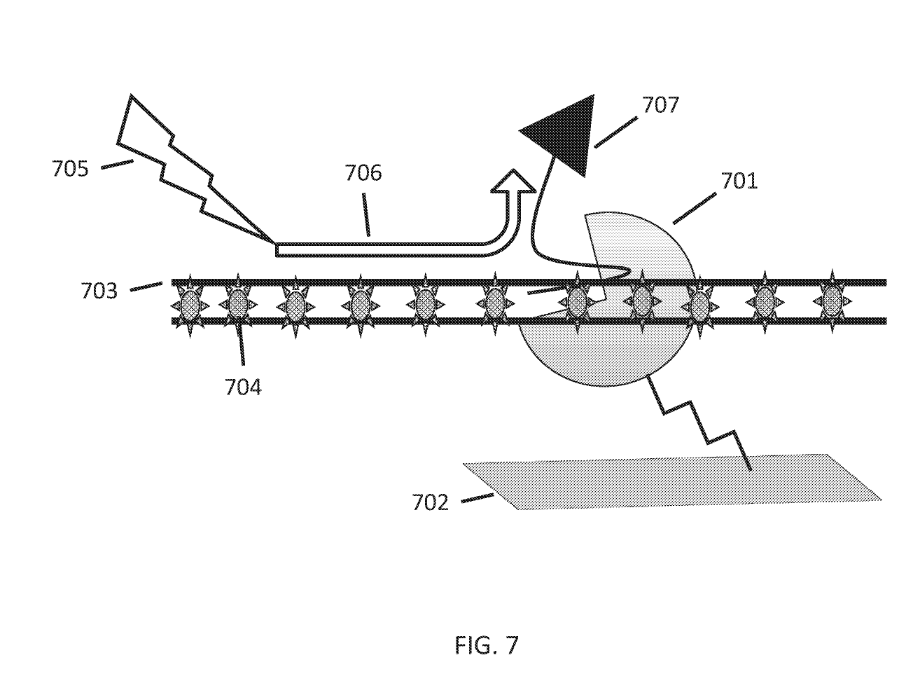

FIG. 7 illustrates an exemplary scheme for utilizing FRET (fluorescence resonance energy transfer) to detect the target nucleic acids (e.g. DNA). In this embodiment labeled target-specific gNA-nucleic acid-guided nuclease system protein complexes 701 are attached to a surface 702. DNA 703 is then allowed to bind the complexes. Unbound DNA is washed off. An intercalating dye 704 such as YOYO-1 is associated with the DNA which can transfer energy 705 via FRET to the label 707 on the complexes. This energy transfer 706 can also occur if two complexes come into close proximity; for example, when two catalytically dead nucleic acid-guided nuclease system (e.g., dCas9) proteins are targeted to the same local region, they could be in close enough proximity to act as FRET pairs. In another embodiment, the labeled gNA-nucleic acid-guided nuclease complexes bound near the end of a DNA fragment can FRET transfer energy to a label on the end of another fragment.

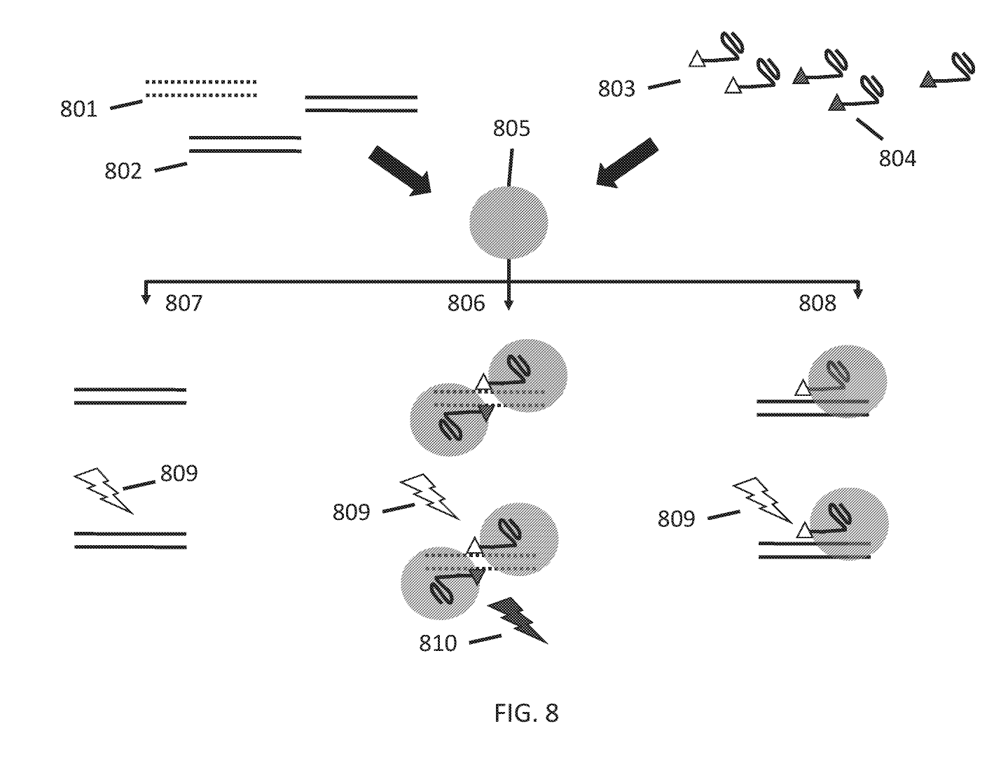

In another exemplary embodiment (FIG. 8), target-specific gNAs are designed and generated in pairs that bind closely on the target (e.g., less than 10 bp apart) and are labeled separately each with one of a FRET pair of fluorophores. A sample can contain target nucleic acids (e.g. DNA) 801 and non-target nucleic acids (e.g. DNA) 802. A first subset of the gNAs 803 are labeled with a first fluorophore, a donor fluorophore for FRET, and a second subset of the gNAs 804 are labeled with a second fluorophore, the acceptor fluorophore. For example, a FRET pair can comprise the labels Alexa Fluor 594 and Alexa Fluor 647, respectively. In an example, the gNAs are complexed to a catalytically inactive nucleic acid-guided nuclease system protein 805 (for example, dCas9) and combined with the DNA sample. When two gNAs bind target DNA in close proximity 806, the donor and acceptor fluorophore are brought in sufficient proximity to allow for FRET. The dead nucleic acid-guided nuclease system-gNA complex binds the target DNA in proximal pairs, allowing for FRET between the first and second fluorophore pairs to occur. Samples are then illuminated with the donor fluorophore excitation wavelength 809 and emission is monitored at the acceptor emission wavelength 810. The FRET signal reflects the presence of target DNA in the sample. In the absence of gNA binding to target 809, no acceptor emission wavelength signal is generated. Non-specific single gNA binding and reaction solution alone will not generate a FRET signal. Rare, non-specific binding of dead nucleic acid-guided nuclease system-guide NAs to non-related targets 808 would result in only a single gNA binding to DNA, thus not resulting in FRET. Similarly, fluorophores in the solution would not be in close proximity continuously thus no FRET signal (or no appreciable FRET signal) from the reaction solution would occur. Thus, such an assay can be carried out in a one-tube format. Such an assay can be useful for applications including detection of target DNA in a low concentration sample (e.g., <5% target DNA).

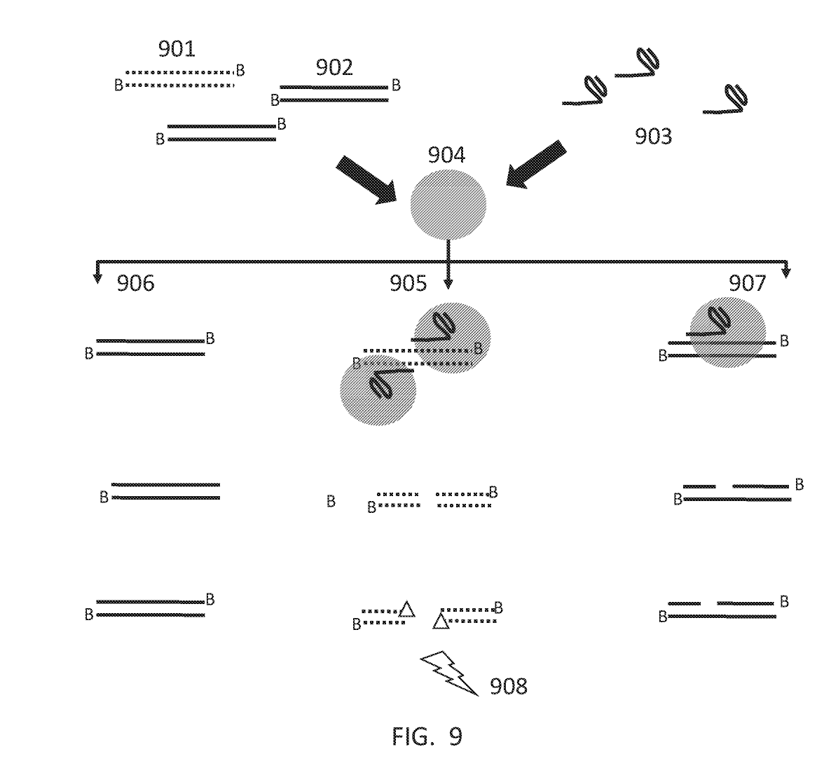

FIG. 9 illustrates a scheme for fluorescent labeling of target nucleic acids (e.g. DNA) using nickase nucleic acid-guided nuclease (e.g., nickase Cas9). In an example (see, e.g., FIG. 9), target-specific gNAs 903 are designed to bind target DNA 901 in pairs in close proximity (e.g., separated by less than ˜10 bp). The sample can also contain non-target DNA 902. The gNAs are complexed to nucleic acid-guided nuclease system nickase protein 904 and combined with the DNA sample. An exemplary nucleic acid-guided nuclease system nickase protein is nickase Cas9, comprising a D10A mutant, which is only capable of cutting one strand of the DNA, thus resulting in a nick rather than a double-strand break. In this example, the 3′ ends of the DNA samples are blocked with non-extendable nucleotides (‘B’ in the figure), such as dideoxy CTP. The DNA is recovered from the reaction solution. The nickase-gNA complex can generate two proximal nicks on the target DNA, thus generating a double stranded break 905. When two nicks are in close proximity (e.g., ˜10 bp or less), a double stranded break is produced. These double stranded breaks are the only substrate for end labeling with a fluorophore using terminal transferase. Samples are then incubated with terminal transferase and fluorescently labeled dCTP. Non-target DNA can remain un-nicked 906. Rare, non-specific binding of nickase-guide NA complexes to non-related targets 907 would result in only a single guide binding to DNA, thus resulting in a single nick to the DNA, which would not a substrate for a terminal transferase. Fluorescence 908 can be measured, enabling identification and quantification of target DNA in the sample. The DNA can be recovered using a DNA cleaning and concentration kit (e.g. Zymo research).

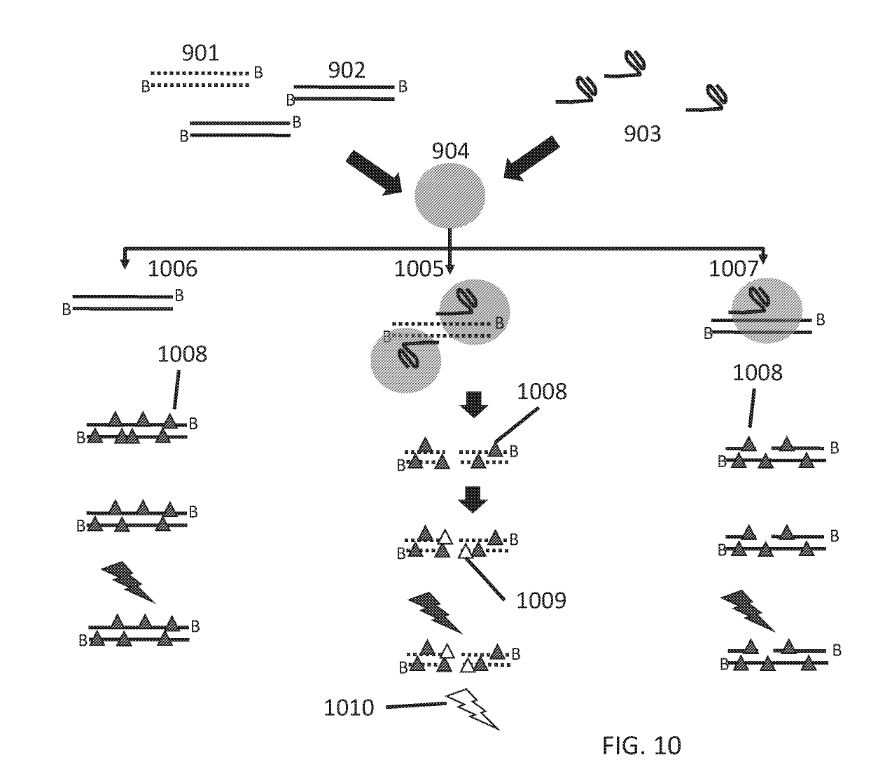

In some embodiments (see, e.g., FIG. 10), target-specific gNAs 903 are designed to bind target nucleic acids (e.g. DNA) 901 in pairs in close proximity (e.g., separated by less than ˜10 bp). The sample can also contain non-target DNA 902. The 3′ ends of the DNA samples are blocked with a non-extendable nucleotide, such as dideoxy CTP (‘B’ in the figure). These gNAs are complexed with a nucleic acid-guided nuclease system nickase protein 904 and added to the DNA sample, resulting in nicks in the DNA at all binding sites for the nickase-gNA complexes. When two nicks are in close proximity (e.g., about ˜10 bp or less), a double stranded break is produced 1005. The entire DNA sample is labeled with a DNA binding dye 1008 that will act as the fluorescent donor. The double stranded breaks, however, are the only substrates for end labeling with an acceptor fluorophore 1009, for example by using terminal transferase. The close proximity of the acceptor label introduced at target sites only to the donor label introduced over the entire DNA results in FRET and emission of the acceptor fluorophore 1010, allowing quantitation of the target DNA in a DNA sample. In the absence of gNA binding to target 1006, no nicking occurs and so no acceptor fluorophore is attached. Rare, non-specific binding of gNA-nickase complexes to non-related targets 1007 would result in only a single complex binding to DNA, thus resulting in a single nick to the DNA, and this would not serve as a substrate for terminal transferase. The close proximity of the acceptor label introduced at target sites only to the donor label introduced over the entire DNA results in FRET and emission of the acceptor fluorophore, allowing quantitation of the target DNA in a DNA sample. Rare, non-specific binding of gNA-nickase complexes to non-related targets would result in only a single complex binding to DNA, thus resulting in a single nick to the DNA, and this would not serve as a substrate for terminal transferase. Close proximity of the donor and acceptor fluorophore can result in FRET, which is measured to calculate the quantity of target DNA in the DNA sample. Fluorophores in the solution and donor fluorophore on the DNA would not be in close proximity continuously thus no FRET signal (or negligible FRET signal) from the reaction solution would occur. Thus, this and the other FRET-based embodiments described above could potentially be carried out in a one-tube format without washing, making these approaches advantageous for rapid processing and detection. Such assays can be useful for applications including detection of target DNA in a low concentration sample (e.g., <5% target DNA). Alternative embodiments include similar approaches with acceptor and donor fluorophore positioning swapped.

Nucleic acids can be brought into proximity with a substrate by a variety of means in addition to diffusion. Electrophoresis and/or fluid flow can be used to concentrate nucleic acids at or near a surface. Other techniques can also be employed. For example, a surface can have hydrophobic surface chemistry over all or some of its surface (e.g., at features), and target nucleic acids can be tagged with a hydrophobic moiety, leading the nucleic acids to have an energetic preference for the hydrophobic regions of the surface. In another example, target nucleic acids can be tagged with a magnetic particle, and magnetic fields can be used to bring the target nucleic acids toward an array surface.

Volume-excluding compounds can also be used to effectively concentrate sample nucleic acids, such as sample DNA. A volume excluder can be used to exclude sample material from the liquid volume occupied by the volume excluder, thereby concentrating the sample material in the remaining liquid volume. This mechanism can help accelerate capture or binding of sample material, such as hybridization of sample nucleic acids to a substrate. For example, volume excluders can be included in a hybridization buffer to improve hybridization kinetics. Volume excluders can be, for example, beads or polymers, including but not limited to dextran sulfate, ficoll, and polyethylene glycol. Volume excluders can be high molecular weight polymers. Volume excluders can be negatively charged, for example to reduce binding of nucleic acids to the volume excluders.

### Kits and Articles of Manufacture

The present application provides kits comprising any one or more of the compositions and collections described herein, not limited to target-specific gNAs, a collection of target-specific gNAs, labeled target-specific gNAs, target-specific gNAs complexed with nucleic acid-guided nuclease system proteins (e.g., CRISPR/Cas system proteins), target-specific gNAs attached to a substrate, target-specific gNAs complexed with nucleic acid-guided nuclease system proteins (e.g., CRISPR/Cas system proteins) and attached to a substrate, pathogen-specific gNAs, a collection of pathogen-specific gNAs, labeled pathogen-specific gNAs, pathogen-specific gNAs complexed with nucleic acid-guided nuclease system proteins (e.g., CRISPR/Cas system proteins), pathogen-specific gNAs attached to a substrate, pathogen-specific gNAs complexed with nucleic acid-guided nuclease system proteins (e.g., CRISPR/Cas system proteins) and attached to a substrate, substrates, and the like. In one embodiment, the nucleic acid-guided nuclease system protein is Cas9.

The present application also provides for compositions and kits with all essential reagents and instructions for carrying out the methods of making, labeling, or attaching the target-specific gNAs and the target-specific gNA-nucleic acid-guided nuclease system protein complexes (e.g., gNA-nucleic acid-guided nuclease system protein complexes) to substrates, as described herein. Reagents can include dyes, and fluorescent nucleotides necessary for detection.

Also provided herein is computer software monitoring the information before and after carrying the methods of detection and identification provided herein.

The following examples are included for illustrative purposes and are not intend to limit the scope of the invention.

### EXAMPLES

### Example 1: Detection of a Pathogen in a Clinical Sample

In this example, a clinical sample (e.g., blood, swab, cerebrospinal fluid) is obtained and DNA or RNA is extracted (e.g., using a Qiagen Blood DNA or RNA extraction kit). If RNA is extracted, it is converted into cDNA using standard methods. Optionally, the DNA can be sheared using enzymatic or mechanical means. The DNA is then applied to a substrate that contains pathogen-specific gRNAs complexed with dCas9 in a known specific order. A colorimetric or fluorescent readout determines the type of pathogen present (e.g., Ebola, HIV, Mycobacterium tuberculosis, etc.). The specific gRNA library used in the detector can be tailored to detect pathogens in different scenarios (e.g, pneumonia, urinary tract infection, foodborne infections). The rapid readout of the detector shortens the time between sample collection and diagnosis compared to standard methods such as culturing or RT-PCR, and detection can be carried out on-site—for example, in the field at the site of an outbreak. The target DNA can also be eluted from the substrate and subsequently sequenced to obtain additional information about the pathogen.

### Example 2: Detection of a Bioweapon (Pathogen) in an Environmental Sample

In this example, an environmental sample (e.g., air filter, soil sample, surface swab) is obtained and DNA or RNA is extracted (e.g., using a MO Bio Soil DNA extraction kit). If RNA is extracted, it is converted into cDNA using standard methods. Optionally, the DNA can be sheared using enzymatic or mechanical means. The DNA is then applied to a substrate, which contains gRNAs targeting a set of potential bioweapon agents, and the colorimetric or fluorescent readout determines the type of pathogenic bioweapon present (e.g., Bacillus anthraces, Yersinia pestis). The rapid readout of the detector shortens the time between sample collection and detection of a threat compared to standard methods such as RT-PCR, and detection can be carried out on-site (e.g., in the field). The target DNA can also be eluted from the substrate and subsequently sequenced to obtain additional information about the pathogen.

### Example 3: Toxic Plant Origin

In this example, a sample of a bioweapon toxin is obtained, and DNA or RNA is extracted (e.g., using a MO Bio Soil DNA extraction kit). If RNA is extracted, it is converted into cDNA using standard methods. Optionally, the DNA can be sheared using enzymatic or mechanical means. The DNA is then applied to a substrate, which contains gRNAs targeting a set of potential toxin agents. For example, if the toxin were ricin, then subspecies of the castor plant itself could allow rapid identification of the toxin, as well as possible subspecies and region of origin of the castor plant.

### Example 4: Product Verification and Barcoding

Product-specific gRNAs could be made to identify different lots or origins of materials such that the origin of a product could be tracked. For example, a baked good could contain ingredients from multiple countries of origin, or a beef sample could be contaminated with horsemeat. An array of location-specific gRNAs could direct enforcement agencies to the location of the bulk ingredients for a product. It could also be helpful in the detection of counterfeit goods that are smuggled into the country by identifying DNA markers on the product packaging.

### Example 5: Human Identification

In this example, a sample containing human DNA (e.g., forensic sample) is obtained and DNA is extracted (e.g., using a QIAmp DNA Micro kit kit). Optionally, the DNA can be sheared using enzymatic or mechanical means. The DNA is then applied to a substrate, which contains gRNAs targeting a set of SNPs selected for human identification, and the colorimetric or fluorescent readout can be selected so as to be unique for each individual. The gRNAs would be selected such that different SNP alleles would produce differential binding of the gRNAs. The rapid readout of the detector shortens the time between sample collection and suspect identification compared to standard methods such as PCR and capillary electrophoresis, and detection can be carried out on-site (e.g., in the field). The target DNA can also be eluted from the substrate and subsequently sequenced to obtain additional information about the individual (e.g., phenotype information).

### Example 6: Detection of Mutations of Tumor DNA

In this example, a sample containing cancer DNA (e.g., a tumor sample) is obtained and DNA is extracted (e.g., using a Qiagen DNeasy tissue kit). Optionally, the DNA can be sheared using enzymatic or mechanical means. The DNA is then applied to a substrate, which contains gRNAs targeting a set of sites commonly mutated in cancers, and the colorimetric or fluorescent readout can be selected so as to be unique for each mutation. The gRNAs would be selected such that different SNP alleles would produce differential binding of the gRNAs. The rapid readout of the detector shortens the time between sample collection and tumor profiling compared to standard methods such as exome sequencing, and detection can be carried out on-site (e.g., in the clinic). The target DNA can also be eluted from the substrate and subsequently sequenced to obtain additional information.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A method of identifying a target in a sample comprising:
a. contacting nucleic acid from a sample with a plurality of guide nucleic acid (gNA)-nucleic acid-guided nuclease system protein complexes, wherein the complexes are targeted to at least one target, and wherein the nucleic acid, the gNA-nucleic acid-guided nuclease system protein complexes, or both the nucleic acid and the gNA-nucleic acid-guided nuclease system protein complexes comprise a label; and
b. identifying the target in the sample, wherein the identifying is achieved by detecting a specific signal from the label, wherein the presence of a specific signal indicates binding of the gNA-nucleic acid-guided nuclease system protein complex to the nucleic acid.

2. The method of claim 1, wherein the nucleic acid comprises a label.

3. The method of claim 2, wherein the label is an intercalating label.

4. The method of claim 2, wherein the nucleic acid is labeled prior to the contacting step.

5. The method of claim 2, wherein the nucleic acid is labeled after the contacting step.

6. The method of claim 2, wherein the nucleic acid-guided nuclease system protein is a CRISPR/Cas system protein.

7. The method of any of claims 1-6, wherein the method further comprises generating droplets, wherein a subset of the droplets comprise a gNA-nucleic acid-guided nuclease system protein complex.

8. The method of claim 7, wherein a subset of the droplets comprise a gNA-nucleic acid-guided nuclease system protein complex bound to the nucleic acid.

9. The method of claim 1, wherein a plurality of the gNA-nucleic acid-guided nuclease system protein complexes comprise a label.

10. The method of claim 9, wherein a plurality of the gNA-nucleic acid-guided nuclease system protein complexes are labeled prior to the contacting step.

11. The method of claim 9, wherein a plurality of the gNA-nucleic acid-guided nuclease system protein complexes are labeled after the contacting step.

12. The method of claim 9, wherein the nucleic acid comprises a label.

13. The method of claim 12, wherein the nucleic acid and the plurality of the gNA-nucleic acid-guided nuclease system protein complexes are both labeled prior to the contacting step.

14. The method of claim 12, wherein the nucleic acid and the plurality of the gNA-nucleic acid-guided nuclease system protein complexes are both labeled after the contacting step.

15. The method of claim 12, wherein the nucleic acid is labeled prior to contacting, and the plurality of the gNA-nucleic acid-guided nuclease system protein complexes are labeled after the contacting step.

16. The method of claim 12, wherein the nucleic acid is labeled after contacting, and the plurality of the gNA-nucleic acid-guided nuclease system protein complexes are labeled before the contacting step.

17. The method of claim 12, wherein the nucleic acid comprises a first label and the gNA-nucleic acid-guided nuclease system protein complexes comprise a second label, wherein the first and second label comprise a donor/acceptor pair for fluorescent resonance energy transfer.

18. The method of any one of claims 1-17, wherein the contacting is carried out at room temperature.

19. The method of any one of claims 1-18, wherein the identifying comprises detecting a single signal

20. The method of any one of claims 1-18, wherein the identifying comprises detecting multiple signals.

21. The method of any one of claims 1-20, wherein the nucleic acid-guided nuclease system protein is a CRISPR/Cas system protein selected from the group consisting of Cas9, CasX, CasY, Cpf1, Cas3, Cas8a-c, Cas10, Cse1, Csy1, Csn2, Cas4, Csm2, and Cm5.

22. The method of claim 21, wherein the nucleic acid-guided nuclease system protein is a dead nucleic acid-guided nuclease system protein.

23. The method of claim 22, wherein the nucleic acid-guided nuclease system protein is dCas9.

24. The method of claim 21, wherein the nucleic acid-guided nuclease system protein is a nucleic acid-guided nuclease system nickase protein.

25. The method of claim 21, wherein the nucleic acid-guided nuclease system protein exhibits reduced off-target binding.

26. The method of any one of claims 1-25, wherein the label is selected from the group consisting of an enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group, an aptamer, one member of a binding pair, and combinations thereof.

27. The method of any one of claims 1-25, wherein the label is a detectable label.

28. The method of any one of claims 1-27, wherein the sample is selected from the group consisting of a clinical sample, a forensic sample, an environmental sample, a metagenomic sample, and a food sample.

29. The method of any one of claims 1-28, wherein the sample is from a human.

30. The method of any one of claims 1-29, wherein the sample is not processed prior to the contacting.

31. The method of any one of claims 1-30, wherein the nucleic acid, the gNA-nucleic acid-guided nuclease system protein complexes, or both the nucleic acid and the gNA-nucleic acid-guided nuclease system protein complexes comprise multiple labels.

32. The method of any one of claims 1-31, wherein the complexes are targeted to a plurality of targets.

33. The method of any one of claims 1-32, wherein the target is an individual.

34. The method of any one of claims 1-32, wherein the target is a pathogen.

35. The method of claim 33, wherein the pathogen is selected from the pathogens in Table 1.

36. The method of claim 32, wherein the complexes targeted to different targets comprise different labels.

37. The method of claim 32, wherein the complexes targeted to different targets comprise the same label.

38. The method of any one of claims 1-37, wherein the gNA-nucleic acid-guided nuclease system protein complexes are attached to a substrate.

39. The method of claim 38, wherein the substrate is silica, plastic, glass, or metal.

40. The method of claim 39, wherein the substrate is a 2-dimensional substrate.

41. The method of claim 39, wherein the substrate comprises a 3-dimensional substrate.

42. The method of claim 39, wherein the substrate comprises a chamber or is a cylindrical array.

43. The method of claim 38, wherein the gNA-nucleic acid-guided nuclease system protein complexes are attached to a substrate in a known order.

44. The method of any one of claims 38-43, wherein the substrate is reusable.

45. The method of any one of claims 1-37, wherein the gNA-nucleic acid-guided nuclease system protein complexes are in solution.

46. The method of any one of claims 1-45, wherein the gNA-nucleic acid-guided nuclease system protein complexes comprise at least 1 unique gNA.

47. The method of any one of claims 1-37, wherein the contacting takes place in solution.

48. The method of any one of claims 1-37, wherein the nucleic acid is attached to a substrate.

49. The method of claim 48, wherein the substrate is silica, plastic, glass, or metal.

50. The method of any one of claims 1-49, wherein the nucleic acid is further analyzed by sequencing.

51. The method of any one of claims 1-50, wherein the nucleic acid is sheared.

52. The method of any one of claims 1-51, wherein the nucleic acid is amplified.

53. The method of any one of claims 1-52, wherein the nucleic acid is blocked at one or more termini with non-extendible nucleotides.

54. The method of any one of claims 1-53, wherein a plurality of targets are simultaneously identified.

55. The method of any one of claims 1-54, wherein the method is carried out in less than 24 hours.

56. The method of any one of claims 1-55, wherein the sample is subject to a prior screening step.

57. The method of any one of claims 1-55, wherein the sample is subject to multi-detection steps.

58. The method of any of claims 1-57, wherein the binding of a gNA-nucleic acid-guided nuclease system protein complex to the nucleic acid indicates the presence of a target.

59. The method of any of claims 1-57, wherein the lack of binding of a gNA-nucleic acid-guided nuclease system protein complex to a nucleic acid indicates the absence of a target.

60. The method of any of claims 1-59, wherein the nucleic acid comprises DNA.

61. The method of any of claims 1-59, wherein the nucleic acid is DNA.

62. A collection of guide nucleic acids (gNAs) targeted to at least one target, wherein a plurality of the gNAs comprise a label.

63. The collection of claim 62, wherein the collection is targeted to a plurality of targets.

64. The collection of any one of claims 62-63, wherein the target is an individual.

65. The collection of any one of claims 62-63, wherein the target is a pathogen.

66. The collection of claim 65, wherein the pathogen is selected from the pathogens in Table 1.

67. The collection of any one of claims 63-66, wherein the gNAs targeted to different targets comprise different labels.

68. The collection of any one of claims 63-66, wherein the gNAs targeted to different targets comprise the same labels.

69. The collection of any one of claims 62-68, wherein the label is a detectable label.

70. The collection of any one of claims 62-69, wherein the label is selected from the group consisting of an enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group, an aptamer, one member of a binding pair, and combinations thereof.

71. The collection of any one of claims 62-70, wherein the gNAs comprise more than one label.

72. The collection of any one of claims 62-71, wherein the gNAs are attached to a substrate.

73. The collection of claim 72, wherein the substrate is silica, plastic, glass, or metal.

74. The collection of claim 73, wherein the substrate is a 2-dimensional substrate.

75. The collection of claim 73, wherein the substrate comprises a 3-dimensional substrate.

76. The collection of claim 73, wherein the substrate comprises a chamber or is a cylindrical array.

77. The collection of any one of claims 62-76, wherein the gNAs are attached to a substrate in a known order.

78. The collection of any one of claims 72-77, wherein the substrate is reusable.

79. The collection of any one of claims 62-71, wherein the gNAs are in solution.

80. The collection of any one of claims 62-79, wherein the collection comprises at least 1 unique gNA.

81. The collection of any one of claims 62-80, wherein the gNAs are complexed to nucleic acid-guided nuclease system proteins.

82. The collection of claim 81, wherein the nucleic acid-guided nuclease system proteins are CRISPR/Cas system proteins.

83. The collection of claim 82, wherein the CRISPR/Cas system proteins are selected from the group consisting of Cas9, CasX, CasY, Cpf1, Cas3, Cas8a-c, Cas10, Cse1, Csy1, Csn2, Cas4, Csm2, and Cm5.

84. The collection of claim 81, wherein the nucleic acid-guided nuclease system protein is a dead nucleic acid-guided nuclease system protein.

85. The collection of claim 84, wherein the nucleic acid-guided nuclease system protein is dCas9.

86. The collection of claim 81, wherein the nucleic acid-guided nuclease system protein is nucleic acid-guided nuclease system nickase protein.

87. The collection of claim 81, wherein the nucleic acid-guided nuclease system protein exhibits reduced off-target binding.

88. The collection of any one of claims 62-87, wherein the nucleic acid-guided nuclease system proteins further comprise a label.

89. A collection of guide nucleic acid (gNA)-nucleic acid-guided nuclease system protein complexes, wherein the gNAs are targeted to at least one target, and wherein a plurality of the complexes comprise a label.

90. The collection of claim 89, wherein the nucleic acid-guided nuclease system protein is a CRISPR/Cas system protein.

91. The collection of claim 89, wherein a plurality of the gNAs comprise a label.

92. The collection of claim 89, wherein a plurality of nucleic acid-guided nuclease system proteins comprise a label.

93. The collection of claim 89, wherein both a plurality of gNAs and a plurality of nucleic acid-guided nuclease system proteins comprise a label.

94. The collection of any one of claims 89-93, wherein the gNAs are targeted a plurality of targets.

95. The collection of any one of claims 89-94, wherein the target is an individual.

96. The collection of any one of claims 89-94, wherein the target is a pathogen.

97. The collection of claim 96, wherein the pathogen is selected from the pathogens in Table 1.

98. The collection of any one of claims 89-97, wherein the gNAs targeted to different targets comprise different labels.

99. The collection of any one of claims 89-97, wherein the gNAs targeted to different targets comprise the same label.

100. The collection of any one of claims 89-97, wherein the nucleic acid-guided nuclease system proteins comprise different labels.

101. The collection of any one of claims 89-97, wherein the nucleic acid-guided nuclease system proteins comprise the same label.

102. The collection of any one of claims 89-101, wherein the label is a detectable label.

103. The collection of any one of claims 89-101, wherein the label is selected from the group consisting of an enzyme, an enzyme substrate, an antibody, an antigen binding fragment, a peptide, a chromophore, a lumiphore, a fluorophore, a chromogen, a hapten, an antigen, a radioactive isotope, a magnetic particle, a metal nanoparticle, a redox active marker group, an aptamer, one member of a binding pair, and combinations thereof.

104. The collection of any one of claims 89-103, wherein the complexes comprise more than one label.

105. The collection of any one of claims 89-104, wherein the complexes are attached to a substrate.

106. The collection of claim 105, wherein the gNAs are attached to the substrate.

107. The collection of claim 105, wherein the nucleic acid-guided nuclease system proteins are attached to the substrate.

108. The collection of claim 105, wherein the substrate is silica, plastic, glass, or metal.

109. The collection of claim 108, wherein the substrate is a 2-dimensional substrate.

110. The collection of claim 108, wherein the substrate is a 3-dimensional substrate.

111. The collection of claim 108, wherein the substrate comprises a chamber or is a cylindrical array.

112. The collection of any one of claims 89-105, wherein the complexes are attached to a substrate in a known order.

113. The collection of any one of claims 89-112, wherein the substrate is reusable.

114. The collection of any one of claims 89-104, wherein the complexes are in solution.

115. The collection of any one of claims 89-114, wherein the collection comprises at least 1 unique gNA.

116. The collection of any one of claims 89-115, wherein the nucleic acid-guided nuclease system protein is a CRISPR/Cas system protein selected from the group consisting of Cas9, CasX, CasY, Cpf1, Cas3, Cas8a-c, Cas10, Cse1, Csy1, Csn2, Cas4, Csm2, and Cm5.

117. The collection of claim 116, wherein the nucleic acid-guided nuclease system protein is a dead nucleic acid-guided nuclease system protein.

118. The collection of claim 117, wherein the nucleic acid-guided nuclease system protein is dCas9.

119. The collection of claim 116, wherein the nucleic acid-guided nuclease system protein is a nucleic acid-guided nuclease system nickase protein.

120. The collection of claim 116, wherein the nucleic acid-guided nuclease system protein exhibits reduced off-target binding.

121. A kit comprising anyone of the collections of claims 62 to 120.
